(19) United States
(12) Patent Application Publication
(10) Pub. No.: US 2011/0028455 A1
(43) Pub. Date: Feb. 3, 2011

(54) Indole-substituted 3-cyanopyridines As Kinase Inhibitors

(75) Inventors: Diane Harris Boschelli, New City, NY (US); Yanong Daniel Wang, Warren, NJ (US); Amarnauth Shastrie Prashad, New City, NY (US); Zhong Li, Congers, NY (US); Chuansheng Niu, Cheshire, CT (US)

Correspondence Address: WYETH LLC;PATENT LAW GROUP, 5 GIRALDA FARMS, MADISON, NJ, 07940, US

(73) Assignee: Wyeth LLC, Madison, NJ (US)

(21) Appl. No.: 12/746,855

(22) PCT Filed: Dec. 12, 2008
(86) PCT No.: PCT/US08/86507

§ 371 Date: Oct. 6, 2010

Related U.S. Application Data
(60) Provisional application No. 61/013,462, filed on Dec. 13, 2007.

(51) Int. Cl. A61K 31/551; C07D 401/12; A61K 31/4439; A61P 11/06; A61P 19/02; A61P 17/06; A61P 1/00; C07D 405/14; A61K 31/496; C07D 401/14; A61K 31/4545; C07D 413/14; A61K 31/5355

(52) U.S. Cl. 514/218; 546/277.4; 514/339; 544/364; 514/253.09; 544/373; 514/254.09; 546/194; 514/318; 544/129; 514/235.2; 540/575

(57) ABSTRACT

Disclosed are compounds of formula (I) and pharmaceutically acceptable salts thereof, wherein X is —O—, —N(R³)—, —S—, —S(O)— or —S(O)₂—; R² is a C₁₋₄ alkyl group or —CF₃; and R¹, R³, R⁴ and p are as defined herein; wherein the compounds are useful as kinase inhibitors. Also disclosed are pharmaceutical compositions containing, and intermediate compounds and methods for making the compounds of formula (I) and their pharmaceutically acceptable salts; and methods of using the foregoing to treat inflammatory and autoimmune diseases such as asthma, colitis, multiple sclerosis, psoriasis, arthritis, rheumatoid arthritis, inflammatory bowel disease, and joint inflammation.

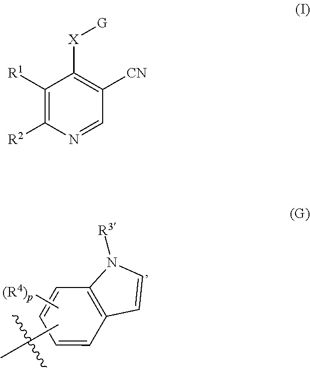

### TECHNICAL FIELD

This invention relates to 3-cyanopyridine compounds which are useful as kinase inhibitors.

### BACKGROUND

Protein kinases are enzymes that catalyze the transfer of a phosphate group from adenosine triphosphate (ATP) to an amino acid residue, such as tyrosine, serine, threonine or histidine, on a protein. Regulation of these protein kinases is essential for the control of a wide variety of cellular events including proliferation and migration. A large number of diseases are associated with abnormal cellular events that are mediated by these kinases, for example, various inflammatory diseases and autoimmune diseases such as asthma, psoriasis, arthritis, rheumatoid arthritis, inflammatory bowel disease, and joint inflammation. See, e.g., Salek-Ardakami, S., et al., J. Immunology, 2004, 173(10), 6440-47; Marsland, B., et al., J. Exp. Med., 2004, 200(2), 181-89; Tan, S, et al., J. Immunology, 2006, 176, 2872-79; Salek-Ardakami, S., et al., J. Immunology, 2005, 175(11), 7635-41; Anderson, K., et al., Autoimmunity, 2006, 39(6), 469-78; Healy, A., et al., J. Immunology, 2006, 177(3), 1886-93; Sun, Z., et al., Nature, 2000, 404, 402-7; and Pfeifhofer, C., et al., J. Exp. Med., 2003, 197(11), 1525-35.

One class of serine/threonine kinases is the protein kinase C (PKC) family. This group of kinases consists of 10 members that share sequence and structural homology. The PKCs are divided into 3 groups and include the classic, the novel, and the atypical isoforms. The theta isoform (PKCθ) is a member of the novel calcium-independent class of PKCs (Baier, G. et al. (1993), J. Biol. Chem., 268: 4997-5004). PKCθ is highly expressed in T cells (Mischak, H. et al. (1993), FEBS Lett., 326: 51-5), with some expression reported in mast cells (Liu, Y. et al. (2001), J. Leukoc. Biol., 69: 831-40), endothelial cells (Mattila, P. et al. (1994), Life Sci., 55: 1253-60), and skeletal muscles (Baier, G. et al. (1994), Eur. J. Biochem., 225: 195-203). It has been shown that PKCθ plays an essential role in T cell receptor (TCR)-mediated signaling (Tan, S. L. et al. (2003), Biochem. J., 376: 545-52). Specifically, it has been observed that inhibiting PKCθ signal transduction, as demonstrated with two independent PKCθ knockout mouse lines, will result in defects in T cell activation and interleukin-2 (IL-2) production (Sun, Z. et al. (2000), Nature, 404: 402-7; Pfeifhofer, C. et al. (2003), J. Exp. Med., 197: 1525-35). It also has been shown that PKCθ-deficient mice show impaired pulmonary inflammation and airway hyperresponsiveness (AHR) in a Th2-dependent murine asthma model, with no defects in viral clearance and Th1-dependent cytotoxic T cell function (Berg-Brown, N. N. et al. (2004), J. Exp. Med., 199: 743-52; Marsland, B. J. et al. (2004), J. Exp. Med., 200: 181-9). The impaired Th2 cell responses result in reduced levels of interleukin-4 (IL-4) and immunoglobulin E (IgE), contributing to the AHR and inflammatory pathophysiology.

Evidence also exists that PKCθ participates in the IgE receptor (FceRI)-mediated response of mast cells (Liu, Y. et al. (2001), J. Leukoc. Biol., 69: 831-840). In human-cultured mast cells (HCMC), it has been demonstrated that PKC kinase activity rapidly localizes (in less than five minutes) to the membrane following FceRI cross-linking (Kimata, M. et al. (1999), Biochem. Biophys. Res. Commun., 257(3): 895-900). A recent study examining in vitro activation of bone marrow mast cells (BMMCs) derived from wild-type and PKCθ-deficient mice shows that upon FceRI cross-linking, BMMCs from PKCθ-deficient mice produced reduced levels of interleukin-6 (IL-6), tumor necrosis factor-alpha (TNFα), and interleukin-13 (IL-13) in comparision with BMMCs from wild-type mice, suggesting a potential role for PKCθ in mast cell cytokine production in addition to T cell activation (Ciarletta, A. B. et al. (2005), poster presentation at the 2005 American Thoracic Society International Conference).

Other serine/threonine kinases include those of the mitogen-activated protein kinase (MAPK) pathway which consists of the MAP kinases (MAPK) (e.g., erk) and the MAPK kinases (MAPKK) (e.g., mek and their substrates). Members of the raf family of kinases phosphorylate residues on mek. The cyclin-dependent kinases (cdks), including cdc2/cyclin B, cdk2/cyclin A, cdk2/cyclin E and cdk4/cyclin D, and others, are serine/threonine kinases that regulate mammalian cell division. Additional serine/threonine kinases include the protein kinases A and B. These kinases, known as PKA or cyclic AMP-dependent protein kinase and PKB (Akt), play key roles in signal transduction pathways.

Tyrosine kinases (TKs) are divided into two classes: the non-transmembrane TKs and transmembrane growth factor receptor TKs (RTKs). Growth factors, such as epidermal growth factor (EGF), bind to the extracellular domain of their partner RTK on the cell surface which activates the RTK, initiating a signal transduction cascade that controls a wide variety of cellular responses. In addition to EGF, there are several other RTKs including FGFR (the receptor for fibroblast growth factor (FGF)); flk-1 (also known as KDR), and flt-1 (the receptors for vascular endothelial growth factor (VEGF)); and PDGFR (the receptor for platelet derived growth factor (PDGF)). Other RTKs include tie-1 and tie-2, colony stimulating factor receptor, the nerve growth factor receptor, and the insulin-like growth factor receptor. In addition to the RTKs there is another family of TKs termed the cytoplasmic protein or non-receptor TKs. The cytoplasmic protein TKs have intrinsic kinase activity, are present in the cytoplasm and nucleus, and participate in diverse signaling pathways. There are a large number of non-receptor TKs including Abl, Jak, Fak, Syk, Zap-70 and Csk, and the Src family of kinases (SFKs) which include Src, Lck, Lyn, Fyn and others.

### SUMMARY

One aspect of the present invention is directed to compounds of formula I:

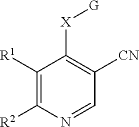

I wherein G is

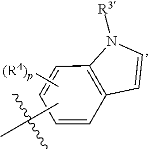

and pharmaceutically acceptable salts thereof, wherein X, R¹, R², R³, R⁴ and p are defined below.

A further aspect of the present invention is directed to compositions comprising a compound of formula I, or a pharmaceutically acceptable salt thereof, and a pharmaceutically acceptable excipient.

A further aspect of the present invention is directed to treating or inhibiting a pathological condition or disorder mediated by a protein kinase (e.g., protein kinase C) in a mammal (e.g., a human), comprising administering to the mammal a therapeutically effective amount of a compound of formula I, or a pharmaceutically acceptable salt thereof.

A further aspect of the present invention is directed to methods of making the compounds of formula I and pharmaceutically acceptable salts thereof.

A further aspect of the present invention is directed to intermediate compounds useful for making the compounds of formula I.

### DEFINITIONS

Except where otherwise indicated, the terms below have the following meanings everywhere in this specification and in the appended claims:

As used herein, the terms “include”, “includes”, “including” and the like are intended to be open-ended unless otherwise specified, i.e., e.g., “including” means “including but not limited to” in the absence of an express limitation.

The use of the singular herein includes the plural (and vice versa) unless specifically stated otherwise. In addition, where the use of the term “about” is before a quantitative value, the present teachings also include the specific quantitative value itself, unless specifically stated otherwise.

As used herein, alone or as part of another group, “halo” or “halogen” refers to fluoro, chloro, bromo and/or iodo.

As used herein, alone or as part of another group, “alkyl” refers to a straight-chain or branched-chain saturated hydrocarbyl group having from 1 to 8 carbon atoms. In some embodiments, an alkyl group can have from 1 to 6 carbon atoms. In some embodiments, an alkyl group can have from 1 to 4 carbon atoms. Examples of C₁₋₄ alkyl groups include methyl, ethyl, propyl, isopropyl, butyl, isobutyl, sec-butyl and tert-butyl. Examples of C₁₋₆ alkyl groups include the aforementioned C₁₋₄ alkyl groups as well as pentyl, isopentyl, neopentyl, hexyl and the like. Additional examples of alkyl groups include heptyl, octyl and the like.

As used herein, alone or as part of another group, “alkylene” refers to a diradical of a straight-chain or branched saturated hydrocarbon group having from 1 to 6 carbon atoms. In some embodiments, an alkylene group can have from 1 to 4 carbon atoms. In some embodiments, an alkylene group can have from 1 to 2 carbon atoms. Examples of C₁₋₂ alkylene groups include methylene and ethylene. Examples of C₁₋₄ alkylene groups include the aforementioned C₁₋₂ alkylene groups as well as trimethylene (1,3-propanediyl), propylene (1,2-propanediyl), tetramethylene (1,4-butanediyl), butylene (1,2-butanediyl), 1,3-butanediyl, 2-methyl-1,3-propanediyl and the like. Examples of C₁₋₆ alkylene groups include the aforementioned C₁₋₄ alkylene groups as well as pentamethylene (1,5-pentanediyl), pentylene (1,2-pentanediyl), hexamethylene (1,6-hexanediyl), hexylene (1,2-hexanediyl), 2,3-dimethyl-1,4-butanediyl and the like. In some embodiments, an alkylene group is an α,ω-diradical. Examples of α,ω-diradical alkylene groups include methylene, ethylene, trimethylene, tetramethylene, pentamethylene and hexamethylene.

As used herein, alone or as part of another group, “alkoxy” or “alkyloxy” refers to an —O-alkyl group having from 1 to 8 carbon atoms. In some embodiments, an alkoxy group can have from 1 to 6 carbon atoms. In some embodiments, an alkoxy group can have from 1 to 4 carbon atoms. Examples of C₁₋₄ alkoxy groups include methoxy, ethoxy, propoxy, isopropoxy, butoxy, tert-butoxy and the like. Examples of C₁₋₆ alkoxy groups include the aforementioned C₁₋₄ alkoxy groups as well as pentyloxy, isopentyloxy, neopentyloxy, hexyloxy and the like. Additional examples of alkoxy groups include heptyloxy, octyloxy and the like.

As used herein, alone or as part of another group, “cycloalkyl” refers to a monocyclic, saturated cyclic hydrocarbyl group having from 3 to 8 ring carbon atoms. In some embodiments (“C₃₋₆ cycloalkyl”), a cycloalkyl group can have from 3 to 6 ring carbon atoms. In some embodiments (“C₅₋₆ cycloalkyl”), a cycloalkyl group can have from 5 to 6 ring carbon atoms. Examples of C₅₋₆ cycloalkyl groups include cyclopentyl and cyclohexyl. Examples of C₃₋₆ cycloalkyl groups include the aforementioned C₅₋₆ cycloalkyl groups as well as cyclopropyl and cyclobutyl.

Examples of C₃₋₈ cycloalkyl groups include the aforementioned C₃₋₆ cycloalkyl groups as well as cycloheptyl and cyclooctyl.

As used herein, alone or as part of another group, “haloalkyl” refers to a alkyl group as defined above wherein one or more of the alkyl group's hydrogen atoms has been replaced with a halogen atom. For each replacement, the halogen atom is independently selected from —F, —Cl, —Br and —I. In some embodiments, a haloalkyl group can be an alkyl group in which one of the alkyl group's hydrogen atoms has been replaced with a halogen atom. Examples of such haloalkyl groups include —CH₂F, —CH₂Cl, —CH₂CH₂Br, —CH₂CH₂I, —CH₂CH₂CH₂F, —CH₂CH₂CH₂Cl, —CH₂CH₂CH₂CH₂Br, —CH₂CH₂CH₂CH₂I, —CH₂CH₂CH₂CH₂CH₂Br, —CH₂CH₂CH₂CH₂CH₂I, —CH₂CH(Br)CH₃, —CH₂CH(Cl)CH₂CH₃, —CH(F)(CH₂)₆CH₃ and —C(C₂H₅)₂(C₂H₄Cl). In some embodiments, a haloalkyl group can be an alkyl group in which one to three of the alkyl group's hydrogen atoms has been replaced with a halogen atom. Examples of such haloalkyl groups include the aforementioned haloalkyl groups as well as —CCl₃, —CF₃ and CH₂CF₃. Other examples of haloalkyl groups include —CF₂(CH₂)₈CHCl₂.

In some embodiments (“perhaloalkyl”), “haloalkyl” can refer to a C₁₋₃ alkyl group wherein all of the hydrogen atoms are each independently replaced with fluoro or chloro. In some embodiments, all of the hydrogen atoms are each replaced with fluoro. In some embodiments, all of the hydrogen atoms are each replaced with chloro. Examples of perhaloalkyl groups include —CF₃, —CF₂CF₃, —CF₂CF₂CF₃, —CCl₃, —CFCl₂, —CF₂Cl and the like.

As used herein, alone or as part of another group, “aryl” refers to a radical of an aromatic monocyclic or bicyclic ring system having from 6 to 10 ring carbon atoms. Examples of such aryl groups include phenyl, 1-naphthyl and 2-naphthyl.

As used herein, alone or as part of another group, “heteroaryl” refers to a radical of a 5- to 10-membered aromatic ring system having ring carbon atoms and 1 to 4 ring heteroatoms, each heteroatom independently selected from N, O and S. Examples of such heteroaryl groups include pyrrolyl, furanyl (furyl), thiophenyl (thienyl), pyrazolyl, imidazolyl, oxazolyl, isoxazolyl, thiazolyl, triazolyl, oxadiazolyl, thiadiazolyl, tetrazolyl, pyridinyl (pyridyl), pyridazinyl, pyrimdinyl, pyrazinyl, triazinyl, indolyl, benzofuranyl, benzothiophenyl (benzothienyl), indazolyl, benzimidazolyl, benzoxazolyl, benzisoxazolyl, benzothiazolyl, quinolinyl, isoquinolinyl, cinnolinyl, quinazolinyl, quinoxalinyl, phthalazinyl, naphthyridinyl and the like. As the foregoing examples illustrate, in some embodiments a heteroaryl group can be monocyclic (“monocyclic heteroaryl”), and in some embodiments a heteroaryl group can be bicyclic (“bicyclic heteroaryl”). In some embodiments (“hetero₁₋₂aryl”), heteroaryl can refer to a heteroaryl group having 1 or 2 ring heteroatoms and otherwise as defined above. In some embodiments (“monocyclic hetero₁₋₂aryl”), heteroaryl can refer to a monocyclic heteroaryl group having 1 or 2 ring heteroatoms and otherwise as defined above. In some embodiments (“bicyclic hetero₁₋₂aryl”), heteroaryl can refer to a bicyclic heteroaryl group having 1 or 2 ring heteroatoms and otherwise as defined above.

As used herein, alone or as part of another group, “heterocyclyl” refers to a radical of a 3- to 10-membered non-aromatic ring system having ring carbon atoms and 1 to 4 ring heteroatoms, each heteroatom independently selected from N, O and S. In some embodiments (“hetero₁₋₃cyclyl”), a heterocyclyl group can have ring atoms selected from carbon atoms and 1 to 3 heteroatoms, and otherwise defined as above. In some embodiments (“hetero₁₋₂cyclyl”), a heterocyclyl group can have ring atoms selected from carbon atoms and 1 or 2 heteroatoms, and otherwise defined as above. Examples of hetero₁₋₂cyclyl groups include pyrrolidinyl, dihydropyrrolyl, tetrahydrofuranyl, dihydrofuranyl, tetrahydrothiophenyl, dihydrothiophenyl, pyrazolidinyl, imidazolidinyl, oxazolidinyl, isoxazolidinyl, thiazolidinyl, piperidinyl, tetrahydropyridinyl, dihydropyridinyl, piperazinyl, tetrahydropyranyl, dioxanyl, morpholinyl, azepanyl, diazepanyl, diazepinyl, oxepanyl, dioxepanyl, oxazepanyl, oxazepinyl and the like. Examples of hetero₁₋₃cyclyl groups include the aforementioned hetero₁₋₂cyclyl groups as well as oxiranyl, aziridinyl, oxetanyl, azetidinyl, triazolidinyl, oxadiazolidinyl, triazinanyl and the like. Additional examples of heterocyclyl groups include decahydroisoquinolinyl, decahydroisoquinolinyl, octahydrochromenyl, octahydroisochromenyl, decahydronaphthyridinyl and the like.

As used herein, alone or as part of another term, “pharmaceutically acceptable” refers to a substance that is acceptable for use in pharmaceutical applications from a toxicological perspective.

### Compounds

The present teachings provide compounds of formula I:

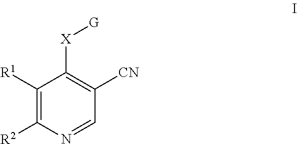

wherein G is

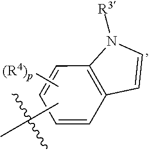

and pharmaceutically acceptable salts thereof, wherein:

### X is —O—, —N(R³)—, —S—, —S(O)— or —S(O)₂—;

R¹ is -A¹-(L)_{q1}-(A²-A³)_{q2}, wherein:
q1 and q2 are each independently 0 or 1;
A¹ is a C₆₋₁₀ aryl group or a 5- to 10-membered hetero₁₋₂aryl group, each of which is optionally substituted with 1 or 2 substituents independently selected from (a) halo, (b) —O—R³, (c) C₁₋₄ alkyl, (d) C₁₋₄ haloalkyl, (f) formyl, (g) —S—R³, (h) —N(R³)R³, (i) -Q-O—R³, (j) -Q-N(R³)—R³, aa) —O-Q-O—R³, (bb) —O-Q-N(R³)R³, (cc) —N(R³)R³-Q-N(R³)R³, (dd) —N(R³)R³-Q-OR³, (p) —C(O)—O—R³, (q) —O—C(O)—R³, (r) —C(O)—N(R³)—R³, (s) —N(R³)—C(O)—R³, (t) —N(R³)—S(O)₂—R³, and (u) —S(O)₂—N(R³)—R³, wherein Q, at each occurrence, is independently C₁₋₄ alkylene;
L is —(Y¹)ₙ₁—Z¹—(Y²)ₙ₂—(Z²)ₙ₃—, wherein:
n1, n2 and n3 are each independently 0 or 1,
provided that when n2 is 0, then n3 is also 0;
Y¹ and Y² are each independently —O—, —N(R³)—, —S—, —S(O)—, —S(O)₂—, —C(O)—O—, —O—C(O)—, —C(O)—N(R³)—, —N(R³)—C(O)—, —N(R³)—S(O)₂—, or —S(O)₂—N(R³)—; and
Z¹ and Z² are each independently C₁₋₄ alkylene;
A² is (a) halo, (b) —O—R³, (g) —S—R³, (h) —N(R³)R³, (k) phenyl, (l) 5- or 6-membered hetero₁₋₂aryl, (m) 3- to 8-membered hetero₁₋₂cyclyl, (ee) Q-(3- to 8-membered hetero₁₋₂cyclyl), (ff) —C(O)-(3- to 8-membered hetero₁₋₂cyclyl), (gg) C₂₋₄ alkene or (hh) C₂₋₄alkyne, wherein each of (k)-(m), (ee)-(hh) is optionally substituted with 1 or 2 substituents independently selected from (a) halo, (b) —O—R³, (c) C₁₋₄ alkyl, (d) C₁₋₄ haloalkyl, (g) —S—R³, (h) —N(R³)R³, (i) -Q-O—R³, (j) -Q-N(R³)—R³, (p) —C(O)—O—R³, (q) —O—C(O)—R³, (r) —C(O)—N(R³)—R³, (s) —N(R³)—C(O)—R³, (t) —N(R³)—S(O)₂—R³, and (u) —S(O)₂—N(R³)—R³, wherein Q, at each occurrence, is independently C₁₋₄ alkylene, and
A³ is (e) H, (k) phenyl, (m) 3- to 8-membered hetero₁₋₂cyclyl, (n) C₃₋₈ cycloalkyl, or (o) an electron pair, wherein each of (k), (m) and (n) is optionally substituted with 1 or 2 substituents independently selected from (a) halo, (b) —O—R³, (c) C₁₋₄ alkyl, (d) C₁₋₄ haloalkyl, (g) —S—R³, (h) —N(R³)R³, (i) -Q-O—R³, (j) -Q-N(R³)—R³, (p) —C(O)—O—R³, (q) —O—C(O)—R³, (r) —C(O)—N(R³)—R³, (s) —N(R³)—C(O)—R³, (t) —N(R³)—S(O)₂—R³, and (u) —S(O)₂—N(R³)—R³, wherein Q, at each occurrence, is independently C₁₋₄ alkylene;
provided that when A² is (a) halo, (b) —O—R³, (g) —S—R³ or (h) —N(R³)R³, then A³ is (o) an electron pair on the halogen atom or heteroatom;
R² is (c) C₁₋₄ alkyl or (d) —CF₃;
   R³, at each occurrence, is independently selected from (e) H and (c) C₁₋₄ alkyl;
R^{3′} is (e) H or (c) C₁₋₄ alkyl;
   R⁴, at each occurrence, is independently selected from (a) halogen, (b) —O—R³, (c) C₁₋₄ alkyl, (d) —CF₃ and (h) —N(R³)—R³; and
p is 0, 1 or 2.

Unless otherwise specified, when a subgroup is designating with a multiple occurrence, each occurrence is selected independently. For example, in —N(R³)—R³,

the R³ groups can be the same or different.

The optionally substituted indolyl group G can be attached to X at the 4-, 5-, 6- or 7-position of the indolyl ring. Each R⁴ group can be attached to any open (i.e., not occupied by X or another R⁴ group) position of the indolyl ring selected from the 2-, 3-, 4-, 5-, 6- or 7-position.

In some embodiments, when q1 is 0 then q2 is also 0.

In some embodiments, X is —O— or —N(R³)—. In some embodiments, X is —N(R³)—. Suitable values of R³ when X is —N(R³)— include H, methyl, ethyl, propyl and butyl; particularly useful values include H, methyl and ethyl, especially H and methyl. In some embodiments, X is —O— or —NH—. In some embodiments, X is —NH—.

In some embodiments, A¹ is a C₆₋₁₀ aryl group optionally substituted with 1 or 2 substituents independently selected from (a) halo, (b) —O—R³, (c) C₁₋₄ alkyl, (d) C₁₋₄ haloalkyl, (f) formyl, (g) —S—R³, (h) —N(R³)R³, (i) -Q-O—R³, (j) -Q-N(R³)—R³, aa) —O-Q-O—R³, (bb) —O-Q-N(R³)R³, (cc) —N(R³)R³-Q-N(R³)R³ and (dd) —N(R³)R³-Q-OR³. In some embodiments, A¹ is a C₆₋₁₀ aryl group not substituted with any of these substituents. In some embodiments, A¹ is a C₆₋₁₀ aryl group substituted with 1 or 2 of these substituents. In some embodiments, A¹ is a C₆₋₁₀ aryl group substituted with 1 of these substituents.

In some embodiments, A¹ is phenyl, substituted or unsubstituted as described in the preceding paragraph.

In some embodiments, A¹ is a 5- to 10-membered hetero₁₋₂aryl group optionally substituted with 1 or 2 substituents independently selected from (a) halo, (b) —O—R³, (c) C₁₋₄ alkyl, (d) C₁₋₄ haloalkyl, (f) formyl, (g) —S—R³, (h) —N(R³)R³, (i) -Q-O—R³ a) -Q-N(R³)—R³, (aa) —O-Q-O—R³, (bb) —O-Q-N(R³)R³, (cc) —N(R³)R³-Q-N(R³)R³ and (dd) —N(R³)R³-Q-OR³. In some embodiments, A¹ is a 5- to 10-membered hetero₁₋₂aryl group not substituted with any of these substituents. In some embodiments, A¹ is a 5- to 10-membered hetero₁₋₂aryl group substituted with 1 or 2 of these substituents. In some embodiments, A¹ is a 5- to 10-membered hetero₁₋₂aryl group substituted with 1 of these substituents.

In some embodiments, A¹ is a bicyclic hetero₁₋₂aryl group substituted or unsubstituted as described in the preceding paragraph. In some embodiments, A¹ is substituted or unsubstituted benzofuranyl, benzothienyl or indolyl. In some embodiments, A¹ is substituted or unsubstituted benzofuranyl.

Suitable values of halo when A¹ is substituted with halo include fluoro, chloro and bromo. Suitable values of R³ when A¹ is substituted with —O—R³, —S—R³, —N(R³)R³, -Q-O—R³ or -Q-N(R³)—R³ include H, methyl, ethyl, propyl, isopropyl, butyl and isobutyl; particularly useful values include H, methyl, ethyl, propyl and isopropyl, especially H, methyl and ethyl. Suitable values of C₁₋₄ alkyl when A¹ is substituted with C₁₋₄ alkyl include methyl, ethyl, propyl, isopropyl, butyl and isobutyl; particularly useful values include methyl, ethyl and propyl, especially methyl and ethyl. Suitable values of C₁₋₄ haloalkyl when A¹ is substituted with C₁₋₄ haloalkyl include —CF₃ and the monochloro and monobromo derivatives of methyl, ethyl, propyl, isopropyl, butyl and isobutyl; particularly useful values include —CF₃ and the monochloro and monobromo derivatives of methyl, ethyl and propyl, especially —CF₃ and the monochloro and monobromo derivatives of methyl and ethyl. Suitable values of Q when A¹ is substituted with -Q-O—R³ or -Q-N(R³)—R³ include methylene, ethylene, trimethylene and tetramethylene; particularly useful values include methylene, ethylene and trimethylene, especially methylene and ethylene.

In some embodiments, Y¹ and Y² are each independently —O— or —N(R³)—. Suitable values of R³ when Y¹ and/or Y² are —N(R³)— include H, methyl, ethyl, propyl and butyl; particularly useful values include H, methyl and ethyl, especially H and methyl. In some embodiments, Y¹ is —O— or —NH—. In some embodiments, Y² is —O— or —NH—.

Suitable values of Z¹ and Z² include methylene, ethylene, trimethylene and tetramethylene; particularly useful values include methylene, ethylene and trimethylene, especially methylene and ethylene.

In some embodiments, R² is C₁₋₄ alkyl. Suitable values of R² when R² is C₁₋₄ alkyl include methyl, ethyl, propyl, isopropyl, butyl and isobutyl; particularly useful values include methyl, ethyl and propyl, especially methyl and ethyl. In some embodiments, R² is methyl.

Suitable values of R^{3′} include H, methyl, ethyl, propyl, isopropyl, butyl and isobutyl; particularly useful values include H, methyl, ethyl, propyl and isopropyl, especially H, methyl and ethyl. In some embodiments, R^{3′} is H.

Suitable values of R⁴ when R⁴ is halogen include fluoro, chloro and bromo. Suitable values of R⁴ when R⁴ is C₁₋₄ alkyl include methyl, ethyl, propyl, isopropyl, butyl and isobutyl; particularly useful values include methyl, ethyl and propyl, especially methyl and ethyl. Suitable values of R³ when R⁴ is —O—R³ or —N(R³)—R³ include H, methyl, ethyl, propyl, isopropyl, butyl and isobutyl; particularly useful values include H, methyl, ethyl, propyl and isopropyl, especially H, methyl and ethyl. In some embodiments, R⁴ is fluoro, chloro, bromo, methyl, ethyl, —CF₃, —O—R³ or —N(R³)—R³, wherein each occurrence of R³ is independently selected from H, methyl and ethyl. In some embodiments, R⁴ is C₁₋₄ alkyl. In some embodiments, R⁴ is methyl.

In some embodiments, G is

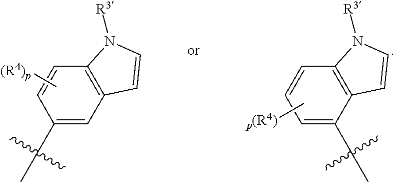

In some embodiments, G is indol-5-yl, 2-methylindol-5-yl, 4-methylindol-5-yl, 7-chloro-4-methyl-5-yl or indol-4-yl. In some embodiments, G is indol-5-yl or 4-methylindol-5-yl.

In some embodiments, p is 0. In some embodiments, p is 1 or 2. In some embodiments, p is 1.

In some embodiments, n2 and n3 are each 0; Y¹ is —O— or —N(R³)—; and Z¹ is methylene, ethylene or trimethylene.

In some embodiments, n2 and n3 are each 1; Y¹ is —O— or —N(R³)—; Z¹ is methylene, ethylene or trimethylene; Y² is —O— or —N(R³)—; and Z² is methylene, ethylene or trimethylene.

In some embodiments, q1 and q2 are each 1; and n1, n2 and n3 are each 0.

In some embodiments, A² is (a) halo, (b) —O—R³, (k) phenyl, (l) 5- or 6-membered hetero₁₋₂aryl (m) 5- to 7-membered hetero₁₋₂cyclyl, (ee) Q-(3- to 8-membered hetero₁₋₂cyclyl) or (ff) —C(O)-(3- to 8-membered hetero₁₋₂cyclyl), (gg) C₂₋₄ alkene or (hh) C₂₋₄ alkyne wherein each of (k)-(m), (ee)-(hh) is optionally substituted with 1 or 2 substituents—in some embodiments, 1 substituent—independently selected from (a) halo, (b) —O—R³, (c) C₁₋₄ alkyl, (d) C₁₋₄ haloalkyl, (g) —S—R³, (h) —N(R³)R³, (i) -Q-O—R³ and (j) -Q-N(R³)—R³. In some embodiments, A² is (a) halo, (b) —O—R³, (k) phenyl, (l1) imidazolyl, (l2) pyridinyl, (m1) pyrrolidinyl, (m2) piperidinyl, (m3) piperazinyl, (m4) morpholinyl or (m5) 1,4-diazepanyl; wherein each of (k)-(m5), is optionally substituted with 1 or 2 substituents—in some embodiments, 1 substituent—independently selected from (b) —O—R³, (c) C₁₋₄ alkyl, (i) -Q-O—R³ and (j) -Q-N(R³)—R³.

In some embodiments, A³ is (e) H, (k) phenyl, (m) 5- to 7-membered hetero₁₋₂cyclyl, (n) C₅₋₇ cycloalkyl or (o) an electron pair; wherein each of (k), (m) and (n) is optionally substituted with 1 or 2 substituents independently selected from (a) halo, (b) —O—R³, (c) C₁₋₄ alkyl, (d) C₁₋₄ haloalkyl, (g) —S—R³, (h) —N(R³)R³, (i) -Q-O—R³ and (j) -Q-N(R³)—R³. In some embodiments, A³ is (e) H, (k) phenyl, (m1) pyrrolidinyl, (m2) piperidinyl, (m3) morpholinyl, (n) cyclopentyl or (o) an electron pair; wherein each of (k), (m) and (n) is independently optionally substituted with 1 or 2 substituents independently selected from (b) —O—R³, (c) C₁₋₄ alkyl, (i) -Q-O—R³ and (j) -Q-N(R³)—R³.

Suitable values of halo when A² or A³ is substituted with halo include fluoro, chloro and bromo. Suitable values of R³ when A² or A³ is substituted with —O—R³, —S—R³, —N(R³)R³, -Q-O—R³ or -Q-N(R³)—R³ include H, methyl, ethyl, propyl, isopropyl, butyl and isobutyl; particularly useful values include H, methyl, ethyl, propyl and isopropyl, especially H, methyl and ethyl. Suitable values of C₁₋₄ alkyl when A² or A³ is substituted with C₁₋₄ alkyl include methyl, ethyl, propyl, isopropyl, butyl and isobutyl; particularly useful values include methyl, ethyl and propyl, especially methyl and ethyl. Suitable values of C₁₋₄ haloalkyl when A² or A³ is substituted with C₁₋₄ haloalkyl include —CF₃ and the monochloro and monobromo derivatives of methyl, ethyl, propyl, isopropyl, butyl and isobutyl; particularly useful values include —CF₃ and the monochloro and monobromo derivatives of methyl, ethyl and propyl, especially —CF₃ and the monochloro and monobromo derivatives of methyl and ethyl. Suitable values of Q when A² or A³ is substituted with -Q-O—R³ or -Q-N(R³)—R³ include methylene, ethylene, trimethylene and tetramethylene; particularly useful values include methylene, ethylene and trimethylene, especially methylene and ethylene.

Illustrative compounds of the invention include those in the following table.

| Name | Structure |
|---|---|
| Example 8 5-(3,4-dimethoxyphenyl)-4-(1H- indol-4-ylamino)-6- methylnicotinonitrile | 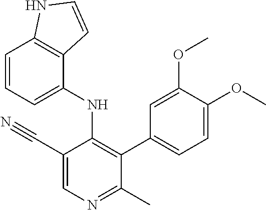 |
| Example 9 5-(3,4-dimethoxyphenyl)-6-methyl- 4-[(4-methyl-1H-indol-5- yl)amino]nicotinonitrile | 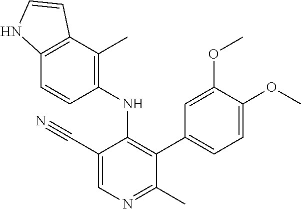 |
| Example 10 5-(1-benzofuran-2-yl)-4-(1H-indol- 4-ylamino)-6-methylnicotinonitrile | 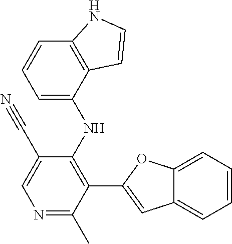 |
| Example 11 5-(1-benzofuran-2-yl)-6-methyl-4- [(4-methyl-1H-indol-5- yl)amino]nicotinonitrile | 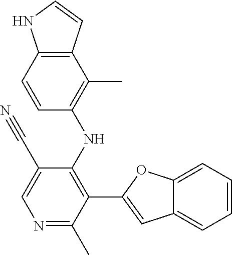 |
| Example 13 5-[4-(2-chloroethoxy)phenyl]-4- (1H-indol-4-ylamino)-6- methylnicotinonitrile | 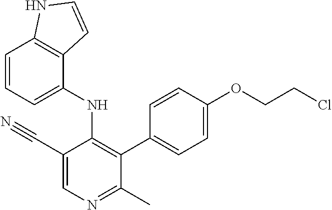 |
| Example 12 5-[4-(2-chloroethoxy)phenyl)-6- methyl-4-[(4-methyl-1H-indol-5- yl)amino]nicotinonitrile | 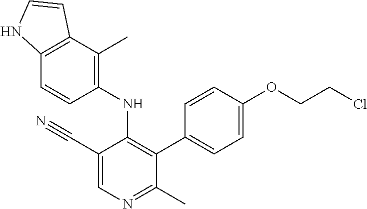 |
| Example 14 6-methyl-4-[(4-methyl-1H-indol-5- yl)amino]-5-{4-[2-(4- methylpiperazin-1- yl)ethoxy]phenyl}nicotinonitrile | 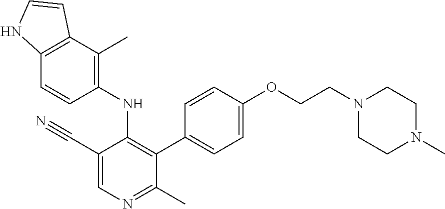 |
| Example 15 5-(5-formyl-1-benzofuran-2-yl)-6- methyl-4-[(4-methyl-1H-indol-5- yl)amino]nicotinonitrile | 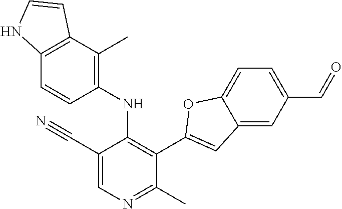 |
| Example 16 6-methyl-4-[(4-methyl-1H-indol-5- yl)amino]-5-{5-[(4-methylpiperazin- 1-yl)methyl]-1-benzofuran-2- yl}nicotinonitrile | 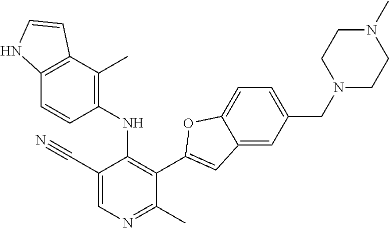 |
| Example 17 5-(4-{2-[(2-hydroxyethyl)- amino]ethoxy}phenyl)-6-methyl-4- [(4-methyl-1H-indol-5- yl)amino]nicotinonitrile | 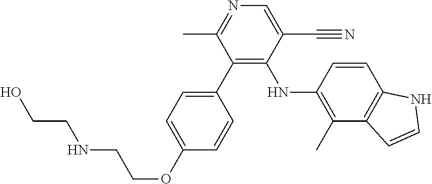 |
| Example 18 5-(4-{2-[(3-hydroxypropyl)- amino]ethoxy}phenyl)-6-methyl-4- [(4-methyl-1H-indol-5-yl)amino] nicotinonitrile | 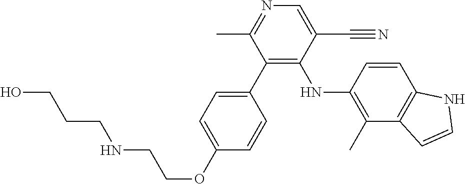 |
| Example 19 5-(4-{2-[(2-ethoxyethyl)amino]- ethoxy}phenyl)-6-methyl-4-[(4- methyl-1H-indol-5- yl)amino]nicotinonitrile | 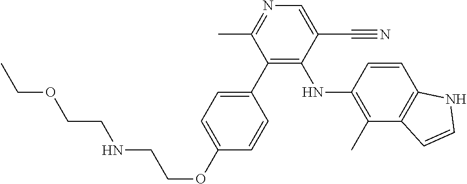 |
| Example 20 6-methyl-4-[(4-methyl-1H-indol-5- yl)amino]-5-[4-(2-pyrrolidin-1- ylethoxy)phenyl]nicotinonitrile | 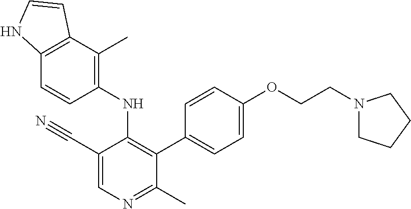 |
| Example 21 6-methyl-4-[(4-methyl-1H-indol-5- yl)amino]-5-(4-{2-[(2-pyrrolidin-1- ylethyl)amino]-ethoxy}phenyl)- nicotinonitrile | 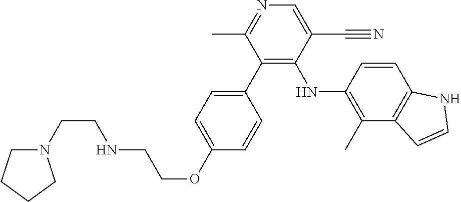 |
| Example 22 6-methyl-4-[(4-methyl-1H-indol-5- yl)amino]-5-(4-{2-[(1-methyl- piperidin-4-yl)amino]- ethoxy}phenyl)nicotinonitrile | 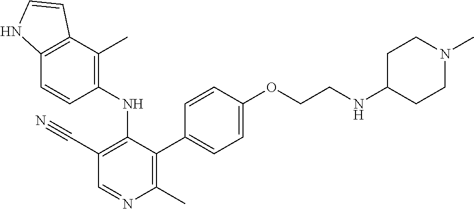 |
| Example 23 6-methyl-4-[(4-methyl-1H-indol-5- yl)amino]-5-[4-(2-{[(1- methylpiperidin-4- yl)methyl]amino}ethoxy)- phenyl]nicotinonitrile | 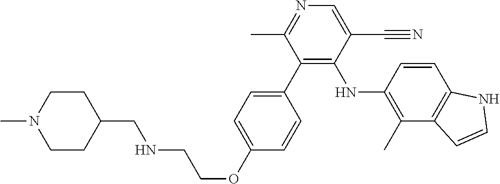 |
| Example 24 6-methyl-4-[(4-methyl-1H-indol-5- yl)amino]-5-[4-(2-piperidin-1- ylethoxy)phenyl]nicotinonitrile | 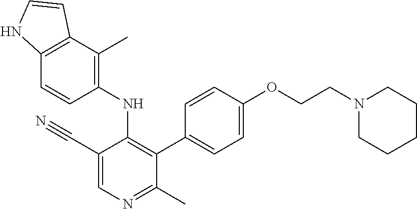 |
| Example 25 5-(4-{2-[4-(2- hydroxyethyl)piperidin-1- yl]ethoxy}phenyl)-6-methyl-4-[(4- methyl-1H-indol-5- yl)amino]nicotinonitrile | 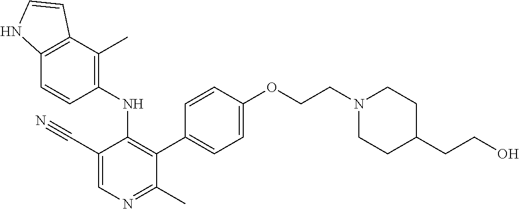 |
| Example 26 6-methyl-4-[(4-methyl-1H-indol-5- yl)amino]-5-{4-[2-(4-pyrrolidin-1- ylpiperidin-1-yl)ethoxy]- phenyl}nicotinonitrile | 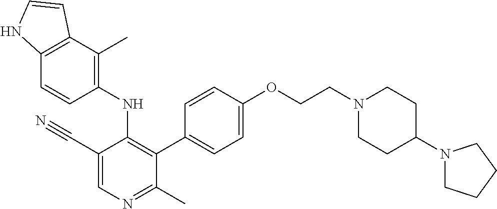 |
| Example 27 5-{4-[2-(1,4′-bipiperidin-1′- yl)ethoxy]phenyl}-6-methyl-4-[(4- methyl-1H-indol-5- yl)amino]nicotinonitrile | 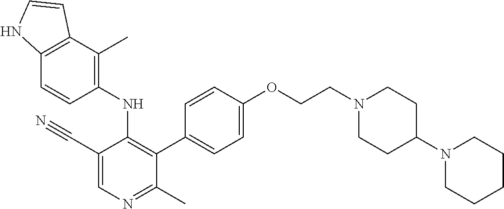 |
| Example 28 6-methyl-4-[(4-methyl-1H-indol-5- yl)amino]-5-{4-[2-(4-morpholin-4- ylpiperidin-1- yl)ethoxy]phenyl}nicotinonitrile | 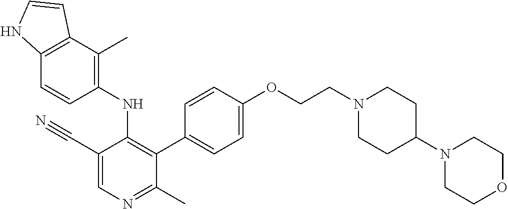 |
| Example 29 6-methyl-4-[(4-methyl-1H-indol-5- yl)amino]-5-{4-[2-(4- phenylpiperidin-1- yl)ethoxy]phenyl}nicotinonitrile | 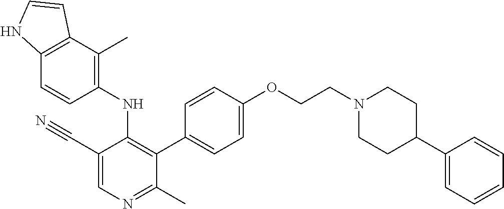 |
| Example 30 6-methyl-4-[(4-methyl-1H-indol-5- yl)amino]-5-[4-(2-morpholin-4- ylethoxy)phenyl]nicotinonitrile | 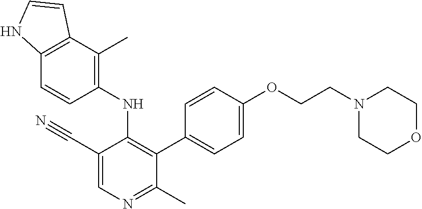 |
| Example 31 5-{4-[2-(2,5-dimethylpiperazin-1- yl)ethoxy]phenyl}-6-methyl-4-[(4- methyl-1H-indol-5- yl)amino]nicotinonitrile | 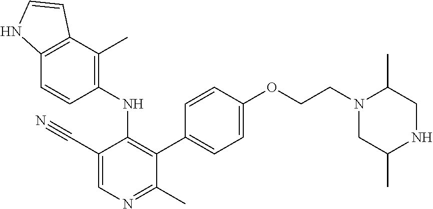 |
| Example 32 5-{4-[2-(3,5-dimethylpiperazin-1- yl)ethoxy]phenyl}-6-methyl-4-[(4- methyl-1H-indol-5- yl)amino]nicotinonitrile | 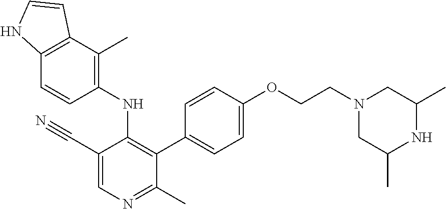 |
| Example 33 5-{4-[2-(4-ethylpiperazin-1- yl)ethoxy]phenyl}-6-methyl-4-[(4- methyl-1H-indol-5- yl)amino]nicotinonitrile | 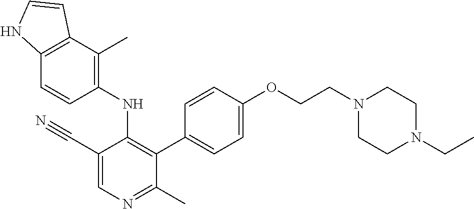 |
| Example 34 5-{4-[2-(1,4-diazepan-1- yl)ethoxy]phenyl}-6-methyl-4-[(4- methyl-1H-indol-5- yl)amino]nicotinonitrile | 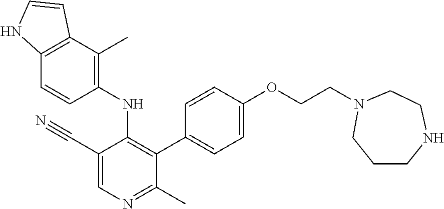 |
| Example 35 5-(4-{2-[4-(2- hydroxyethyl)piperazin-1- yl]ethoxy}phenyl)-6-methyl-4-[(4- methyl-1H-indol-5- yl)amino]nicotinonitrile | 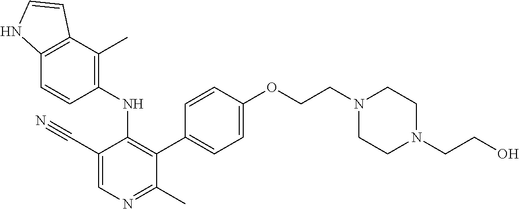 |
| Example 36 5-[4-(2-{4-[2-(dimethylamino)- ethyl]piperazin-1-yl}ethoxy)- phenyl]-6-methyl-4-[(4-methyl-1H- indol-5-yl)amino]nicotinonitrile | 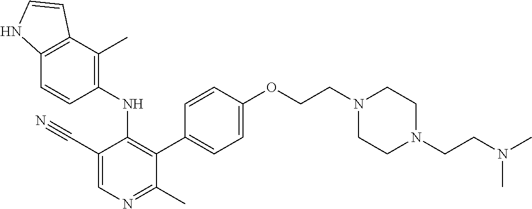 |
| Example 37 5-{4-[2-(4-cyclopentylpiperazin-1- yl)ethoxy]phenyl}-6-methyl-4-[(4- methyl-1H-indol-5- yl)amino]nicotinonitrile | 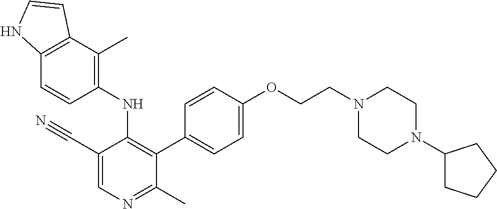 |
| Example 38 5-[4-(2-{[3-(1H-imidazol-1- yl)propyl]amino}ethoxy)phenyl]-6- methyl-4-[(4-methyl-1H-indol-5- yl)amino]nicotinonitrile | 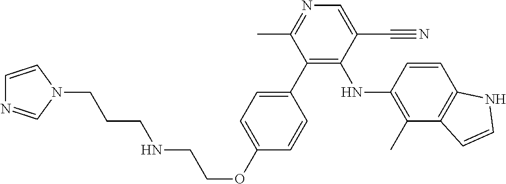 |
| Example 39 5-{4-[2-(benzylamino)ethoxy]- phenyl}-6-methyl-4-[(4-methyl-1H- indol-5-yl)amino]nicotinonitrile | 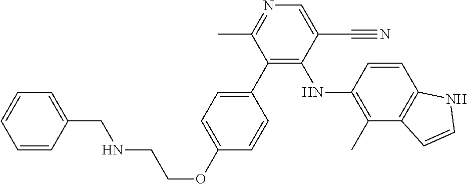 |
| Example 40 6-methyl-4-[(4-methyl-1H-indol-5- yl)amino]-5-(4-{2-[(pyridin-2- ylmethyl)amino]ethoxy}phenyl) nicotinonitrile | 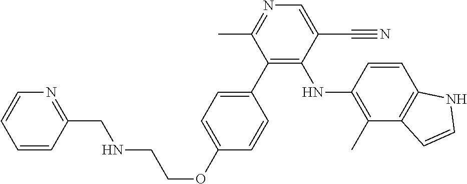 |
| Example 41 6-methyl-4-[(4-methyl-1H-indol-5- yl)amino]-5-(4-{2-[(pyridin-3- ylmethyl)amino]ethoxy}phenyl) nicotinonitrile | 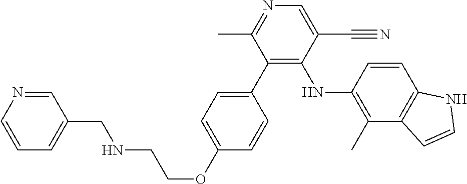 |
| Example 42 6-methyl-4-[(4-methyl-1H-indol-5- yl)amino]-5-(4-{2-[(pyridin-4- ylmethyl)amino]ethoxy}phenyl) nicotinonitrile | 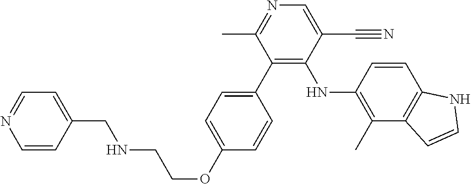 |
| Example 46 5-(3,4-dimethoxyphenyl)-4-(1H- indol-6-ylamino)-6- methylnicotinonitrile | 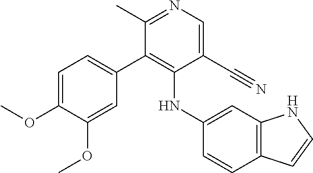 |
| Example 48 5-(3,4-dimethoxyphenyl)-6-methyl- 4-[(2-methyl-1H-indol-5- yl)amino]nicotinonitrile | 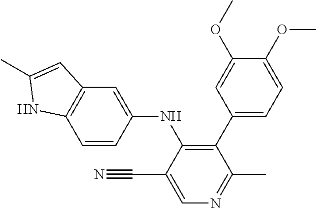 |
| Example 50 4-(1H-indol-5-ylamino)-6-methyl-5- phenylnicotinonitrile | 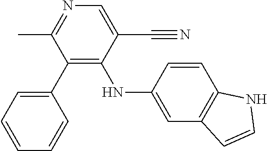 |
| Example 51 5-(3,4-dimethoxyphenyl)-4-(1H- indol-5-ylamino)-6- methylnicotinonitrile | 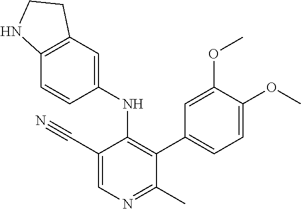 |
| Example 52 4-(1H-indol-5-ylamino)-5-(2- methoxyphenyl)-6- methylnicotinonitrile | 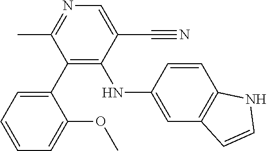 |
| Example 53 4-(1H-indol-5-ylamino)-5-(3- methoxyphenyl)-6- methylnicotinonitrile | 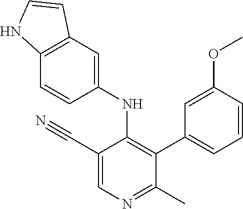 |
| Example 54 4-(1H-indol-5-ylamino)-5-(4- methoxyphenyl)-6- methylnicotinonitrile | 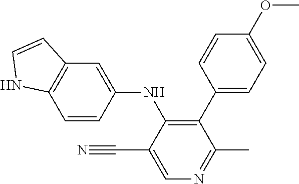 |
| Example 55 5-(1-benzofuran-2-yl)-4-(1H-indol- 5-ylamino)-6-methylnicotinonitrile | 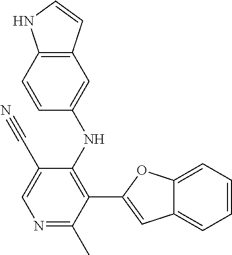 |
| Example 56 5-(1-benzothiophen-2-yl)-4-(1H- indol-5-ylamino)-6- methylnicotinonitrile | 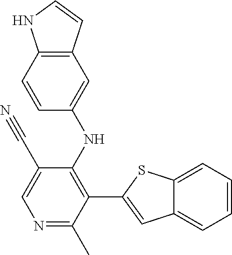 |
| Example 57 5-{3-[(dimethylamino)methyl]- phenyl}-4-(1H-indol-5-ylamino)-6- methylnicotinonitrile | 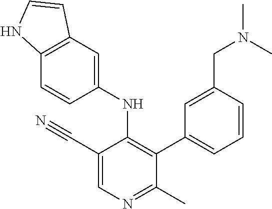 |
| Example 61 5-(3,4-dimethoxyphenyl)-6-ethyl-4- [(4-methyl-1H-indol-5- yl)amino]nicotinonitrile | 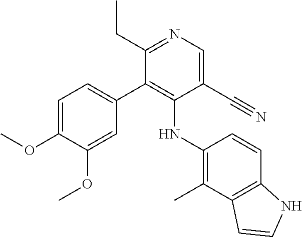 |
| Example 64 4-[(7-chloro-4-methyl-1H-indol-5- yl)amino]-5-(3,4-dimethoxyphenyl)- 6-methylnicotinonitril | 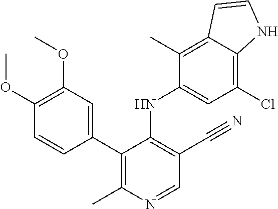 |
| Example 65 4-(1H-indol-5-ylamino)-6-methyl-5- (2-thienyl)nicotinonitrile | 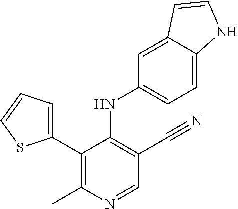 |
| Example 66 5-{3- [(dimethylamino)methyl]phenyl}-6- methyl-4-[(4-methyl-1H-indol-5- yl)amino]pyridine-3-carbonitrile | 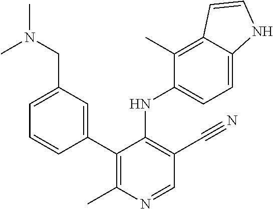 |
| Example 67 5-(5-formylfuran-2-yl)-6-methyl-4- [(4-methyl-1H-indol-5- yl)amino]pyridine-3-carbonitrile | 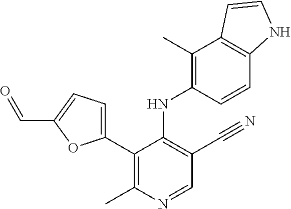 |
| Example 68 6-methyl-4-[(4-methyl-1H-indol-5- yl)amino]-5-{5-[(4-methylpiperazin- 1-yl)methyl]furan-2-yl}pyridine-3- carbonitrile | 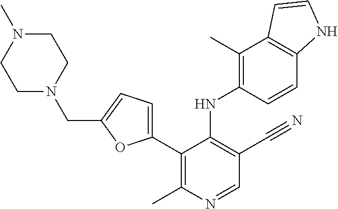 |
| Example 69 5-(5-formylthiophen-2-yl)-6-methyl- 4-[(4-methyl-1H-indol-5- yl)amino]pyridine-3-carbonitrile | 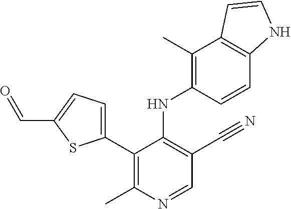 |
| Example 70 6-methyl-4-[(4-methyl-1H-indol-5- yl)amino]-5-{5-[(4-methylpiperazin- 1-yl)methyllthiophen-2-yl}pyridine- 3-carbonitrile | 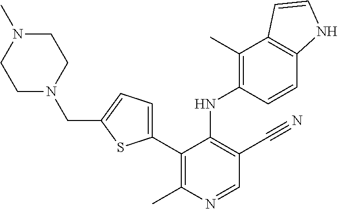 |

Compounds of the present invention may be prepared using intermediates, examples of which include:
4-[(7-chloro-4-methyl-1H-indol-5-yl)amino]-5-iodo-6-methylnicotinonitrile;
5-iodo-6-methyl-4-oxo-1,4-dihydro-pyridine-3-carboxamide;
4-chloro-5-iodo-6-methylnicotinonitrile;
5-bromo-6-methyl-4-oxo-1,4-dihydro-pyridine-3-carboxamide;
5-bromo-4-chloro-6-methylnicotinonitrile;
4-(1H-indol-4-ylamino)-5-iodo-6-methylnicotinonitrile;
5-iodo-6-methyl-4-[(4-methyl-1H-indol-5-yl)amino]nicotinonitrile;
5-bromo-4-(1H-indol-5-ylamino)-6-methylnicotinonitrile;
5-bromo-6-methyl-4-[(4-methyl-1H-indol-5-yl)amino]nicotinonitrile;
5-bromo-4-(1H-indol-6-ylamino)-6-methylnicotinonitrile;
5-iodo-4-(2-methyl-1H-indol-5-ylamino)-6-methylnicotinonitrile;
5-iodo-4-(1H-indol-5-ylamino)-6-methylnicotinonitrile;
4-chloro-6-ethyl-5-iodonicotinonitrile;
5-bromo-4-chloro 6-ethylnicotinonitrile;
6-ethyl-5-iodo-4-(4-methyl-1H-indol-5-ylamino)nicotinonitrile; and,
5-bromo-6-ethyl-4-(4-methyl-1H-indol-5-ylamino)-nicotinonitrile.

Pharmaceutically acceptable salts of the compounds of formula I having an acidic moiety can be formed using organic and/or inorganic bases. Both mono and polyanionic salts are contemplated, depending on the number of acidic hydrogens available for deprotonation. Suitable salts formed with bases include metal salts, such as alkali metal or alkaline earth metal salts, for example sodium, potassium and magnesium salts; ammonia salts and organic amine salts, such as those formed with morpholine, thiomorpholine, piperidine, pyrrolidine, a mono-, di- or tri-(C₁₋₆ alkyl)amine (e.g., ethyl-tert-butyl-, diethyl-, diisopropyl-, triethyl-, tributyl- or dimethylpropyl-amine), or a mono-, di-, or tri-hydroxy (C₁₋₆ alkyl)amine (e.g., mono-, di- or tri-ethanolamine). Examples of inorganic bases useful for forming such pharmaceutically acceptable salts include NaHCO₃, Na₂CO₃, KHCO₃, K₂CO₃, Cs₂CO₃, LiOH, NaOH, KOH, NaH₂PO₄, Na₂HPO₄ and Na₃PO₄. Similarly, pharmaceutically acceptable salts of the compounds of formula I having a basic moiety can be formed using organic and/or inorganic acids. Both mono and polycationic salts are contemplated, depending on the number of lone-pair electrons available for donation. For example, suitable salts can be formed from the following acids: acetic, benzenesulfonic, benzoic, camphorsulfonic, citric, dichloroacetic, ethenesulfonic, formic, fumaric, gluconic, glutamic, hippuric, hydrobromic, hydrochloric, isethionic, lactic, maleic, malic, malonic, mandelic, methanesulfonic, mucic, naphthalenesulfonic, nitric, oxalic, pamoic, pantothenic, phosphoric, phthalic, propionic, succinic, sulfuric, tartaric, and toluenesulfonic.

Esters of the compounds of formula I can include various pharmaceutically acceptable esters known in the art that can be metabolized into the free acid form (e.g., a free carboxylic acid form) in a mammal. Examples of such esters include alkyl esters (e.g., of 1 to 10 carbon atoms), cycloalkyl esters (e.g., of 3-10 carbon atoms), aryl esters (e.g., of 6-14 carbon atoms, including of 6-10 carbon atoms), and heterocyclic analogues thereof (e.g., of 3-14 ring atoms, 1-3 of which can be selected from oxygen, nitrogen, and sulfur heteroatoms), wherein the alcohol residue can include further substituents. In some embodiments, esters of the compounds disclosed herein can be C₁₋₁₀ alkyl esters, such as methyl esters, ethyl esters, propyl esters, isopropyl esters, butyl esters, isobutyl esters, t-butyl esters, pentyl esters, isopentyl esters, neopentyl esters, and hexyl esters; C₃₋₁₀ cycloalkyl esters, such as cyclopropyl esters, cyclopropylmethyl esters, cyclobutyl esters, cyclopentyl esters, and cyclohexyl esters; or aryl esters, such as phenyl esters, benzyl esters, and tolyl esters.

The present teachings also provide compositions that comprise or include at least one compound of formula I, or a pharmaceutically acceptable salt thereof, and one or more pharmaceutically acceptable carriers, excipients, or diluents. Compositions may also consist essentially of one or more compounds of formula I, or a pharmaceutically acceptable salt thereof, together with one or more pharmaceutically acceptable carriers, excipients, or diluents. Suitable excipients are those that do not adversely interact with the active ingredient are compatible with the other ingredients in the formulation and are biologically acceptable. Examples of such excipients are well known to those skilled in the art, e.g., as described in Handbook of Pharmaceutical Excipients, 5th edition, eds. R. C. Rowe, P. J. Sheskey and S. C. Owen, Pharmaceutical Press: London, UK (2003), the entire disclosure of which is incorporated by reference herein. Examples of such compositions are well known to those skilled in the art and can be prepared in accordance with acceptable pharmaceutical procedures, such as, e.g., those described in Remington: The Science and Practice of Pharmacy, 20th edition, ed. A. R. Gennaro, Lippincott Williams & Wilkins: Baltimore, Md. (2000), the entire disclosure of which is incorporated by reference herein. Supplementary active ingredients can also be incorporated into the pharmaceutical compositions.

Compounds of the present teachings can be useful for treating a pathological condition or disorder in a mammal, for example, a human. As used herein, “treating” refers to partially or completely alleviating and/or ameliorating the condition and/or symptoms thereof. The present teachings accordingly include a method of providing to a mammal a pharmaceutical composition that includes a compound of the present teachings in combination or association with a pharmaceutically acceptable carrier. Compounds of the present teachings can be administered alone or in combination with other therapeutically effective compounds or therapies for the treatment of a pathological condition or disorder. As used herein, “therapeutically effective” refers to a substance or an amount that elicits a desirable biological activity or effect.

The present teachings also include use of the compounds disclosed herein as active therapeutic substances for the treatment of a pathological condition or disorder mediated by a protein kinase such as protein kinase C (PKC) and PKC theta isoform (PKCθ). The pathological condition or disorder can include inflammatory diseases and autoimmune diseases such as asthma, colitis, multiple sclerosis, psoriasis, arthritis, rheumatoid arthritis, and joint inflammation. Accordingly, the present teachings further provide methods of treating these pathological conditions and disorders using the compounds described herein. In some embodiments, the methods include identifying a mammal having a pathological condition or disorder mediated by a protein kinase such as PKC and PKCθ, and providing to the mammal an effective amount of a compound as described herein. In some embodiments, the method includes administering to a mammal a pharmaceutical composition that includes a compound disclosed herein in combination or association with a pharmaceutically acceptable carrier.

The present teachings further include use of the compounds disclosed herein as active therapeutic substances for the prevention and/or inhibition of the pathological condition or disorder listed above. Accordingly, the present teachings further provide methods of preventing and/or inhibiting these pathological conditions and disorders using the compounds described herein. In some embodiments, the methods include identifying a mammal having a pathological condition or disorder mediated by a protein kinase such as PKC and PKCθ, and providing to the mammal an effective amount of a compound as described herein. In some embodiments, the method includes administering to a mammal a pharmaceutical composition that includes a compound disclosed herein in combination or association with a pharmaceutically acceptable carrier.

Compounds of the present teachings can be administered orally or parenterally, neat or in combination with conventional pharmaceutical carriers. Applicable solid carriers can include one or more substances which can also act as flavoring agents, lubricants, solubilizers, suspending agents, fillers, glidants, compression aids, binders or tablet-disintegrating agents, or encapsulating materials. The compounds can be formulated in conventional manner, for example, in a manner similar to that used for known antiinflammatory agents. Oral formulations containing an active compound disclosed herein can include any conventionally used oral form, including tablets, capsules, buccal forms, troches, lozenges and oral liquids, suspensions or solutions. In powders, the carrier can be a finely divided solid, which is an admixture with a finely divided active compound. In tablets, an active compound can be mixed with a carrier having the necessary compression properties in suitable proportions and compacted in the shape and size desired. The powders and tablets may contain up to 99% of the active compound.

Capsules can contain mixtures of active compound(s) with inert filler(s) and/or diluent(s) such as the pharmaceutically acceptable starches (e.g., corn, potato or tapioca starch), sugars, artificial sweetening agents, powdered celluloses (e.g., crystalline and microcrystalline celluloses), flours, gelatins, gums, and the like.

Useful tablet formulations can be made by conventional compression, wet granulation or dry granulation methods and utilize pharmaceutically acceptable diluents, binding agents, lubricants, disintegrants, surface modifying agents (including surfactants), suspending or stabilizing agents, including magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes, and ion exchange resins. Preferred surface modifying agents include nonionic and anionic surface modifying agents. Representative examples of surface modifying agents include poloxamer 188, benzalkonium chloride, calcium stearate, cetostearl alcohol, cetomacrogol emulsifying wax, sorbitan esters, colloidal silicon dioxide, phosphates, sodium dodecylsulfate, magnesium aluminum silicate, and triethanolamine. Oral formulations herein can utilize standard delay or time-release formulations to alter the absorption of the active compound(s). The oral formulation can also comprise a compound as described herein in water or fruit juice, containing appropriate solubilizers or emulsifiers as needed.

Liquid carriers can be used in preparing solutions, suspensions, emulsions, syrups, elixirs, and for inhaled delivery. A compound described herein can be dissolved or suspended in a pharmaceutically acceptable liquid carrier such as water, an organic solvent, or a mixture of both, or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers, and osmo-regulators. Examples of liquid carriers for oral and parenteral administration include water (particularly containing additives as described above, e.g., cellulose derivatives such as a sodium carboxymethyl cellulose solution), alcohols (including monohydric alcohols and polyhydric alcohols, e.g., glycols) and their derivatives, and oils (e.g., fractionated coconut oil and arachis oil). For parenteral administration, the carrier can be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellants.

Liquid pharmaceutical compositions, which are sterile solutions or suspensions, can be utilized by, for example, intramuscular, intraperitoneal or subcutaneous injection. Sterile solutions can also be administered intravenously. Compositions for oral administration can be in either liquid or solid form.

Preferably the pharmaceutical composition is in unit dosage form, for example, as tablets, capsules, powders, solutions, suspensions, emulsions, granules, or suppositories. In such form, the pharmaceutical composition can be sub-divided in unit dose(s) containing appropriate quantities of the active compound. The unit dosage forms can be packaged compositions, for example, packeted powders, vials, ampoules, prefilled syringes or sachets containing liquids. Alternatively, the unit dosage form can be a capsule or tablet itself, or it can be the appropriate number of any such compositions in package form. Such unit dosage form may contain from about 1 mg/kg of active compound to about 500 mg/kg of active compound, and can be given in a single dose or in two or more doses. Such doses can be administered in any manner useful in directing the active compound(s) to the recipient's bloodstream, including orally, via implants, parenterally (including intravenous, intraperitoneal and subcutaneous injections), rectally, vaginally, and transdermally. Such administrations can be carried out using the compounds of the present teachings including pharmaceutically acceptable salts thereof, in lotions, creams, foams, patches, suspensions, solutions, and suppositories (rectal and vaginal).

When administered for the treatment or inhibition of a particular disease state or disorder, it is understood that an effective dosage can vary depending upon many factors such as the particular compound utilized, the mode of administration, and severity of the condition being treated, as well as the various physical factors related to the individual being treated. In therapeutic applications, a compound of the present teachings can be provided to a patient already suffering from a disease in an amount sufficient to cure or at least partially ameliorate the symptoms of the disease and its complications. The dosage to be used in the treatment of a specific individual typically must be subjectively determined by the attending physician. The variables involved include the specific condition and its state as well as the size, age and response pattern of the patient.

In some cases, for example those in which the lung is the targeted organ, it may be desirable to administer a compound directly to the airways of the patient, using devices such as metered dose inhalers, breath-operated inhalers, multidose dry-powder inhalers, pumps, squeeze-actuated nebulized spray dispensers, aerosol dispensers, and aerosol nebulizers. For administration by intranasal or intrabronchial inhalation, the compounds of the present teachings can be formulated into a liquid composition, a solid composition, or an aerosol composition. The liquid composition can include, by way of illustration, one or more compounds of the present teachings dissolved, partially dissolved, or suspended in one or more pharmaceutically acceptable solvents and can be administered by, for example, a pump or a squeeze-actuated nebulized spray dispenser. The solvents can be, for example, isotonic saline or bacteriostatic water. The solid composition can be, by way of illustration, a powder preparation including one or more compounds of the present teachings intermixed with lactose or other inert powders that are acceptable for intrabronchial use, and can be administered by, for example, an aerosol dispenser or a device that breaks or punctures a capsule encasing the solid composition and delivers the solid composition for inhalation. The aerosol composition can include, by way of illustration, one or more compounds of the present teachings, propellants, surfactants, and co-solvents, and can be administered by, for example, a metered device. The propellants can be a chlorofluorocarbon (CFC), a hydrofluoroalkane (HFA), or other propellants that are physiologically and environmentally acceptable.

Compounds described herein can be administered parenterally or intraperitoneally. Solutions or suspensions of these active compounds or pharmaceutically acceptable salts, hydrates, or esters thereof can be prepared in water suitably mixed with a surfactant such as hydroxyl-propylcellulose. Dispersions can also be prepared in glycerol, liquid polyethylene glycols, and mixtures thereof in oils. Under ordinary conditions of storage and use, these preparations typically contain a preservative to inhibit the growth of microorganisms.

The pharmaceutical forms suitable for injection can include sterile aqueous solutions or dispersions and sterile powders for the extemporaneous preparation of sterile injectable solutions or dispersions. In preferred embodiments, the form is sterile and its viscosity permits it to flow through a syringe. The form preferably is stable under the conditions of manufacture and storage and can be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

Compounds described herein can be administered transdermally, i.e., administered across the surface of the body and the inner linings of bodily passages including epithelial and mucosal tissues. Such administration can be carried out using the compounds of the present teachings including pharmaceutically acceptable salts, hydrates, and esters thereof, in lotions, creams, foams, patches, suspensions, solutions, and suppositories (rectal and vaginal). Topical formulations that deliver active compound(s) through the epidermis can be useful for localized treatment of inflammation and arthritis.

Transdermal administration can be accomplished through the use of a transdermal patch containing an active compound and a carrier that can be inert to the active compound, can be non-toxic to the skin, and can allow delivery of the active compound for systemic absorption into the blood stream via the skin. The carrier can take any number of forms such as creams and ointments, pastes, gels, and occlusive devices. The creams and ointments can be viscous liquid or semisolid emulsions of either the oil-in-water or water-in-oil type. Pastes comprised of absorptive powders dispersed in petroleum or hydrophilic petroleum containing the active compound can also be suitable. A variety of occlusive devices can be used to release the active compound into the blood stream, such as a semi-permeable membrane covering a reservoir containing the active compound with or without a carrier, or a matrix containing the active compound. Other occlusive devices are known in the literature.

Compounds described herein can be administered rectally or vaginally in the form of a conventional suppository. Suppository formulations can be made from traditional materials, including cocoa butter, with or without the addition of waxes to alter the suppository's melting point, and glycerin. Water-soluble suppository bases, such as polyethylene glycols of various molecular weights, can also be used.

Lipid formulations or nanocapsules can be used to introduce compounds of the present teachings into host cells either in vitro or in vivo. Lipid formulations and nanocapsules can be prepared by methods known in the art.

To increase the effectiveness of compounds of the present teachings, it can be desirable to combine a compound with other agents effective in the treatment of the target disease. For inflammatory diseases, other active compounds (i.e., other active ingredients or agents) effective in their treatment, and particularly in the treatment of asthma and arthritis, can be administered with active compounds of the present teachings. The other agents can be administered at the same time or at different times than the compounds disclosed herein.

Throughout the description, where compositions are described as having, including, or comprising specific components, or where processes are described as having, including, or comprising specific process steps, it is contemplated that compositions of the present teachings also consist essentially of, or consist of, the recited components, and that the processes of the present teachings also consist essentially of, or consist of, the recited processing steps.

It should be understood that the order of steps or order for performing certain actions is immaterial so long as the present teachings remain operable. Moreover, two or more steps or actions may be conducted simultaneously.

Certain intermediate compounds useful for preparing the compounds of formula I are prepared as shown in Scheme 1:

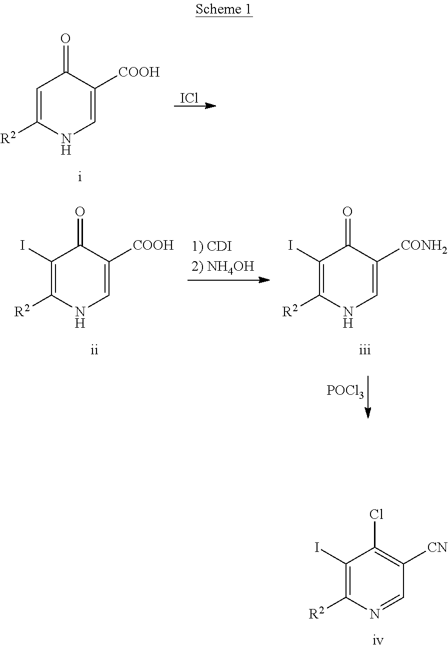

The 6-alkyl-4(1H)-pyridone-3-carboxylic acids i can be iodinated at C-5, for example, by using the method reported in WO9948892 to prepare 5-iodo-6-methyl-4-oxo-1,4-dihydropyridine-3-carboxylic acid (ii where R² is methyl). The carboxylic acid group is converted to the primary amide iii by reaction with N,N-carbonyldiimidazole followed by treatment with aqueous ammonium hydroxide. Heating of the amide iii in phosphorus oxychloride, optionally in the presence of a catalytic amount of DMF, results in dehydration of the amide with concomitant chlorination at C-4 to give the key 4-chloro-5-iodo intermediate iv.

In an analogous fashion, the 5-bromo-4-chloro intermediates can be prepared by the route shown in Scheme 2. The first step in the preparation of these intermediates is bromination of i at C-5 with bromine in acetic acid, containing an optional amount of pyridine.

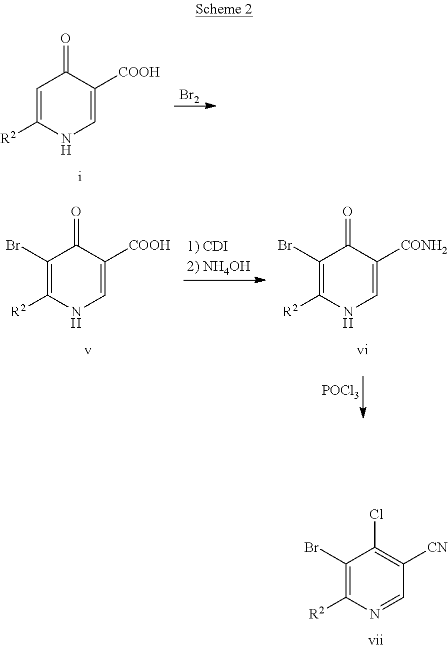

The compounds of formula I can be prepared from intermediates iv and vii as shown in Scheme 3. Reaction of the 4-chloro group of iv or vii with a compound of formula II where X is NR³ gives the 4-amino analog viii where X is NR³. This reaction can be performed in a solvent such as ethanol, propanol, butanol, or 2-ethoxyethanol, at elevated temperatures optionally in the presence of pyridine hydrochloride or triethylamine; or alternatively using an alkali base such as sodium hydride in a solvent such as tetrahydrofuran or dimethylformamide at elevated temperatures. Reaction of the 4-chloro group of iv or vii with a compound of formula II where X is O or S gives the 4-oxygen or sulfur analog viii where X is O or S, respectively. Palladium catalyzed coupling of viii with a boronic acid of formula R¹B(OH)₂, a boronic acid ester of formula R¹B(OR)₂ or a stannane of formula R¹SnR₃ (where R, in each case, is a C₁-C₄alkyl group), provides compounds of formula I. Alternatively, the order of the reactions can be reversed. The intermediates iv and vii can be reacted with a boronic acid of formula R¹B(OH)₂, a boronic acid ester of formula R¹B(OR)₂ or a stannane of formula R¹SnR₃ (where R, in each case, is a C₁-C₄alkyl group), to give compounds of formula ix. Reaction of the 4-chloro group of ix with a compound of formula II gives a compound of formula I.

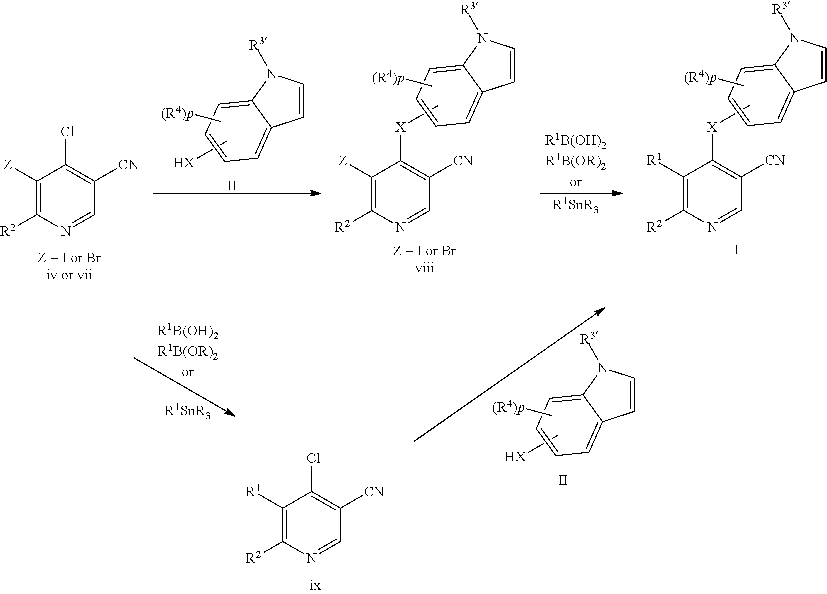

As depicted in Scheme 4, compounds of formula I wherein A¹ is substituted by —CH₂—NR^{a}R^{b} (formula Ib), can be prepared by treating compounds of formula I where A¹ contains an aldehyde functionality (formula Ia), with an amine of formula HNR^{a}R^{b} in the presence of a reducing agent such as sodium triacetoxyborohydride or sodium cyanoborohydride in a solvent such as dichloromethane or THF with the optional addition of DMF or NMP and preferably in the presence of acetic acid. Compounds of formula I wherein A¹ is substituted by —CH₂—OH (formula Ic) can be formed as a byproduct of this reductive amination reaction.

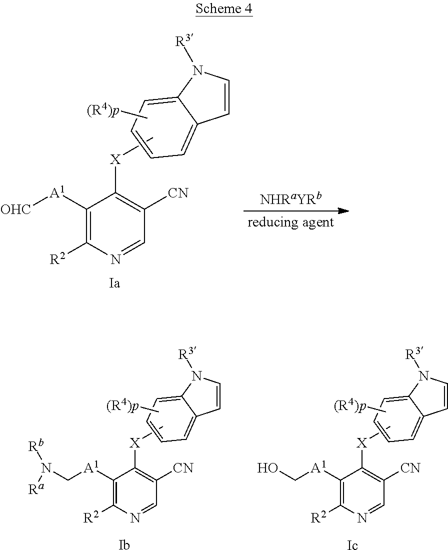

where R^{a} and R^{b} are both R³ or
R^{a} and R^{b} join to form a 3- to 8-membered hetero₁₋₂cyclyl

Referring to Scheme 5 below, compounds of formula I where A¹ is substituted with an A² group selected from an aryl group, a heteroaryl group, an alkenyl group and an alkynyl group (formula Ie) can be prepared from compounds of formula I where A¹ is substituted with a leaving group (LG) such as bromide (Br) or iodide (I) (formula Id). More specifically, compounds of formula Ie where A² is an aryl group or a heteroaryl group can be prepared by treatment of compounds of formula Id with a boronic acid (A²B(OH)₂), a boronic ester (A²B(OR)₂, where R is a C₁-C₄alkyl group) or with an organic stannane reagent (A²SnBu₃) mediated by a palladium catalyst such as (Ph₃P)₄Pd or Pd(OAc)₂) in a solvent such as a mixture of DME and aqueous NaHCO₃ or aqueous. Na₂CO₃, optionally in the presence of a phosphine ligand such as Ph₃P.

Similarly, compounds of formula Ie where A² is an alkenyl group or an alkynyl group can be prepared by treating compounds of formula Id with an alkene or alkyne of formula A²-H or with a boronic acid or ester or an organic stannane reagent in the presence of a palladium catalyst such as (Ph₃P)₄Pd, dichlorobis(triphenyl phosphine)palladium (II), or Pd(OAc)₂) in a solvent such as DMF, NMP, dioxane, or DME, in the presence of a ligand such as Ph₃P or tri-o-tolylphosphine and a base such as potassium carbonate or sodium carbonate, optionally with the addition of an organic base such as triethylamine. A catalytic amount of copper(I) iodide can be optionally used for this coupling reaction.

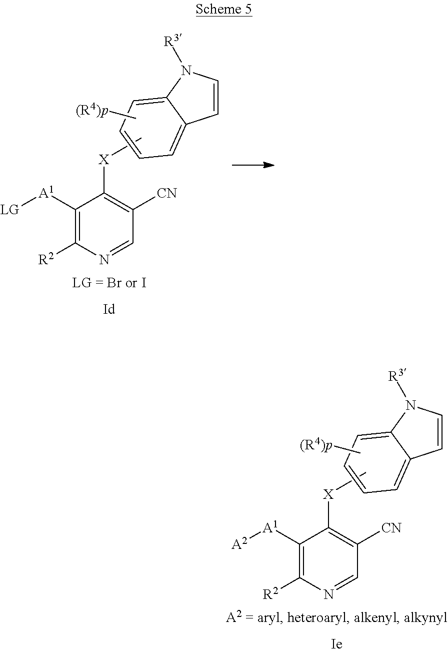

Compounds of formula I where A¹ is substituted with —O—Z¹—NR^{a}R^{b} (formula Ig) can be prepared as depicted in Scheme 6 below, by treating compounds of formula I where A¹ is substituted with —O—Z¹-LG (formula If), where LG is Cl, Br, methanesulfonyl or p-toluenesulfonyl with an amine of formula HNR^{a}R^{b} in a solvent such as EtOH, DME or DMF optionally in the presence of NaI or a base such as K₂CO₃.

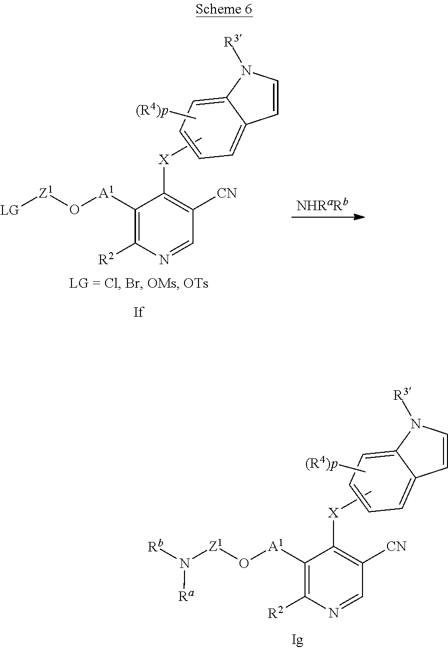

where R^{a} and R^{b} are both R³ or
R^{a} and R^{b} join to form a 3- to 8-membered hetero₁₋₂cyclyl

As depicted in Scheme 7, compounds of formula I where A¹ is substituted by —O—Z¹—(Y²)ₙ₂(Z²)ₙ₃-(A²-A³)_{q2} (formula II) can be prepared by treating compounds of formula I where A¹ contains a hydroxyl functionality (formula Ih), with an alcohol of formula R^{c}OH under Mitsunobu conditions. This reaction can be conducted in a solvent such as THF in the presence of Ph₃P and either diethyl azodicarboxylate or di-t-butyl azodicarboxylate.

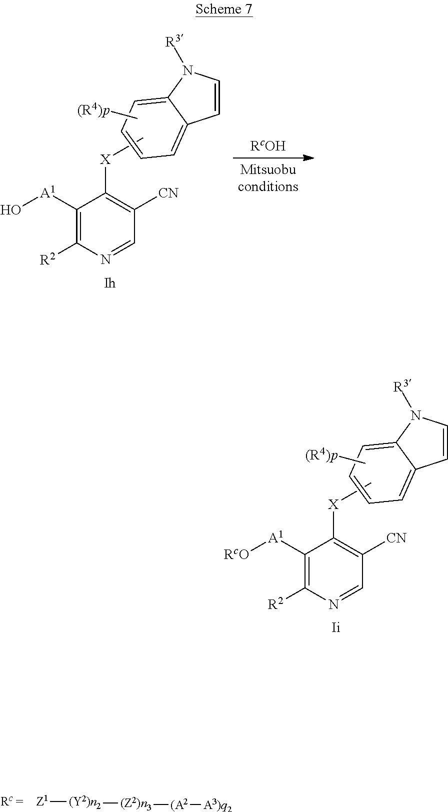

As shown in Scheme 8, treatment of compounds of formula ix with CsF in a solvent such as DMF provides the 4-fluoro analog x. Subsequent displacement of the 4-fluoro group with an amine in a solvent such as DMSO provides compounds of formula I

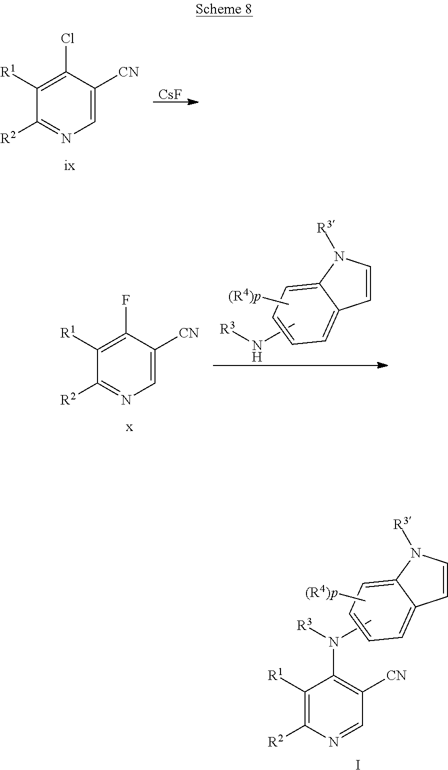

Additional key intermediates can be obtained from compounds of formula iv or vii where R² is methyl. Treatment with a base, such as lithium bis(trimethylsilyl)amide in a solvent such as tetrahydrofuran at reduced temperature, preferably −78° C., followed by addition of an alkyl halide of formula RX provides compounds of formula iv or vii where R² is an extended alkyl group. For example use of iodomethane gives compounds of formula iv or vii where R² is ethyl.

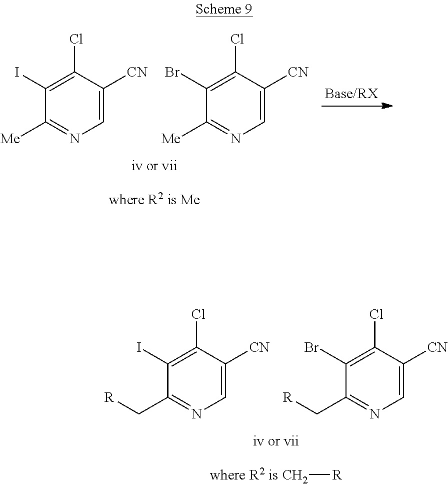

The following examples are presented to illustrate certain embodiments of the present invention, but should not be construed as limiting the scope of this invention.

### EXAMPLE 1

### Preparation of 5-IODO-6-METHYL-4-OXO-1,4-DIHYDRO-PYRIDINE-3-CARBOXAMIDE

6-Methyl-4(1H)-pyridone-3-carboxylic acid was prepared from 4-hydroxy-6-methyl-2-pyrone according to the route published in JOC 37, 1145 (1972). 6-Methyl-4(1H)-pyridone-3-carboxylic acid was converted to 5-iodo-6-methyl-4-oxo-1,4-dihydropyridine-3-carboxylic acid by the route found in WO9948892 step a of Example 82. A mixture of 5-iodo-6-methyl-4-oxo-1,4-dihydropyridine-3-carboxylic acid (5.0 g, 17.9 mmol) and 1,1′-carbonyldiimidazole (CDI) (6.39 g, 39.4 mmol) in 60 mL of DMF was heated at 60-70° C. for 3 h. The reaction mixture was cooled on an ice-bath and poured into cooled 29% aqueous ammonium hydroxide (84 mL). After stirring at 0-5° C. for 3 h, the mixture was poured onto ice and the pH was adjusted to 5-6. The solids were collected by filtration, then washed with water followed by diethyl ether to provide 3.30 g (66%) of 5-iodo-6-methyl-4-oxo-1,4-dihydro-pyridine-3-carboxamide as a white solid, mp 296-298° C.; MS 277.1 (M−H)−.

### EXAMPLE 2

### Preparation of 4-CHLORO-5-IODO-6-METHYLNICOTINONITRILE

A mixture of 5-iodo-6-methyl-4-oxo-1,4-dihydro-pyridine-3-carboxamide (3.0 g, 10.8 mmol) and phosphorus oxychloride (16.6 g, 108 mmol) was heated at 60-70° C. for 30 min. The temperature was slowly increased to 90° C. and a drop of DMF was added. Heating was continued for 2 h resulting in a dark brown solution. The volatiles were removed in vacuo and the residue was slurried with ice. Aqueous sodium bicarbonate was slowly added. The solids were collected by filtration, washing with aqueous sodium bicarbonate and water to provide 2.85 g (95%) of 4-chloro-5-iodo-6-methylnicotinonitrile as a light yellow solid, mp 120-122° C.; MS 279.1 (M+H)⁺.

### EXAMPLE 3

### Preparation of 5-BROMO-6-METHYL-4-OXO-1,4-DIHYDRO-PYRIDINE-3-CARBOXAMIDE

To 6-methyl-4(1H)-pyridone-3-carboxylic acid (1.54 g, 10.06 mmol) and 0.80 mL of pyridine in 30 mL of acetic acid at 100° C. was slowly added 0.72 mL of bromine in 5 mL of acetic acid. The reaction mixture was kept at 100° C. for 2 h then cooled to room temperature. The solids were collected by filtration and washed with methanol to provide 850 mg (36%) of 5-bromo-6-methyl-4-oxo-1,4-dihydro-pyridine-3-carboxamide as a white solid, MS 232.0 (M+H)⁺.

### EXAMPLE 4

### Preparation of 5-BROMO-6-METHYL-4-OXO-1,4-DIHYDRO-PYRIDINE-3-CARBOXAMIDE

A mixture of 5-bromo-6-methyl-4-oxo-1,4-dihydro-pyridine-3-carboxamide (8.0 g, 34.5 mmol) and 1,1′-carbonyldiimidazole (CDI) (12.2 g, 75.3 mmol) in 120 mL of DMF was heated at 65-75° C. for 3 h. The reaction mixture was cooled on an ice-bath and poured into cooled 29% aqueous ammonium hydroxide (84 mL). After stirring at 0-5° C. for 1.5 h, the mixture was poured onto ice and the pH was adjusted to 5-6 with 1 N hydrochloric acid. The solids were collected by filtration, then washed with water followed by diethyl ether to provide 7.25 g (91%) of 5-bromo-6-methyl-4-oxo-1,4-dihydro-pyridine-3-carboxamide as a white solid, MS 228.9(M−H)⁻.

### EXAMPLE 5

### Preparation of 5-BROMO-4-CHLORO-6-METHYLNICOTINONITRILE

A mixture of 5-bromo-6-methyl-4-oxo-1,4-dihydro-pyridine-3-carboxamide (7.12 g, 30.8 mmol) and 1 drop of DMF in 30 mL of phosphorus oxychloride was heated at reflux for 2 h. The volatiles were removed in vacuo and the residue was slurried with ice. Saturated aqueous sodium bicarbonate was slowly added until the mixture had a basic pH. The solids were collected by filtration, washing with aqueous sodium bicarbonate and water to provide 5.50 g (77%) of 5-bromo-4-chloro-6-methylnicotinonitrile as a tan solid, MS 231.0 (M+H)⁺.

### EXAMPLE 6

### Preparation of 4-(1H-INDOL-4-YLAMINO)-5-IODO-6-METHYL-NICOTINONITRILE

A mixture of 4-chloro-5-iodo-6-methylnicotinonitrile (500 mg, 1.80 mmol) and 4-aminoindole (249 mg, 1.89 mmol) in 9 mL of ethanol was heated at reflux overnight. Additional 4-aminoindole (25 mg, 0.189 mmol) was added and the mixture was heated for an additional 24 h. The resulting mixture was cooled to room temperature, diluted with saturated aqueous sodium bicarbonate, filtered and washed with water to give 630 mg (94%) of 4-(1H-indol-4-ylamino)-5-iodo-6-methylnicotinonitrile as a grey solid, mp 192-194° C.; MS 375.0 (M+H).

### EXAMPLE 7

### Preparation of 5-IODO-6-METHYL-4-[(4-METHYL-1H-INDOL-5-YL)AMINO]NICOTINONITRILE

Prepared from 4-chloro-5-iodo-6-methylnicotinonitrile and 5-amino-4-methylindole via the procedure used to prepare 5-iodo-6-methyl-4-[(4-methyl-1H-indol-5-yl)amino]nicotinonitrile, mp 222-223° C.; MS 389.2 (M+H).

### EXAMPLE 8

### Preparation of 5-(3,4-DIMETHOXYPHENYL)-4-(1H-INDOL-4-YLAMINO)-6-METHYLNICOTINONITRILE

A mixture of 5-iodo-6-methyl-4-(1H-indol-4-ylamino)nicotinonitrile (100 mg, 0.267 mmol), 3,4-dimethoxyphenylboronic acid (73 mg, 0.401 mmol) and (Ph₃P)₄Pd (15.4 mg, 0.0134 mmol) in 3 mL of 1,2-dimethoxyethane and 1 mL of saturated aqueous sodium bicarbonate was heated at 90-100° C. for 18 h. After cooling to room temperature the reaction mixture was diluted with aqueous sodium bicarbonate, filtered and washed with water. The crude solid was dissolved in 20 mL of dichloromethane, passed through a short path of Magnesol and washed thoroughly with dichloromethane. The filtrate was concentrated to give 75 mg (74%) of 5-(3,4-dimethoxyphenyl)-4-(1H-indol-4-ylamino)-6-methylnicotinonitrile as an off-white solid, mp 182-183° C.; MS 385.2 (M+H).

### EXAMPLE 9

### Preparation of 5-(3,4-DIMETHOXYPHENYL)-6-METHYL-4-[(4-METHYL-1H-INDOL-5-YL)AMINO]NICOTINONITRILE

Prepared from 5-iodo-6-methyl-4-[(4-methyl-1H-indol-5-yl)amino]nicotinonitrile and 3,4-dimethoxyphenylboronic acid via the procedure used to prepare 5-(3,4-dimethoxy phenyl)-4-(1H-indol-4-ylamino)-6-methylnicotinonitrile, mp 202-203° C.; MS 399.3 (M+H).

### EXAMPLE 10

### Preparation of 5-(1-BENZOFURAN-2-YL)-4-(1H-INDOL-4-YLAMINO)-6-METHYLNICOTINONITRILE

Prepared from 5-iodo-6-methyl-4-(1H-indol-4-ylamino)nicotinonitrile and 2-benzofuranboronic acid via the procedure used to prepare 5-(3,4-dimethoxyphenyl)-4-(1H-indol-4-ylamino)-6-methylnicotinonitrile, mp 192-193° C.; MS 365.2 (M+H).

### EXAMPLE 11

### Preparation of 5-(1-BENZOFURAN-2-YL)-6-METHYL-4-[(4-METHYL-1H-INDOL-5-YL)AMINO]NICOTINONITRILE

Prepared from 5-iodo-6-methyl-4-[(4-methyl-1H-indol-5-yl)amino]nicotinonitrile and 2-benzofuranboronic acid via the procedure used to prepare 5-(3,4-dimethoxyphenyl)-4-(1H-indol-4-ylamino)-6-methylnicotinonitrile, mp 154-155° C.; MS 379.2 (M+H).

### EXAMPLE 12

### Preparation of 5-[4-(2-CHLOROETHOXY)PHENYL]-6-METHYL-4-[(4-METHYL-1H-INDOL-5-YL)AMINO]NICOTINONITRILE

To 4-(2-chloroethoxy)bromobenzene (237 mg, 1.01 mmol) in 5 mL of THF at −78° C. was added tri-isopropyl borate (227 mg, 1.21 mmol) and n-BuLi (1.6 M in hexane, 0.69 mL, 1.11 mmol). After 1 h at −78° C., the reaction mixture was stirred at room temperature overnight, then evaporated to give a solid residue. The residue was mixed with 5-iodo-6-methyl-4-[(4-methyl-1H-indol-5-yl)amino]nicotinonitrile (200 mg, 0.503 mmol) and (Ph₃P)₄Pd in 2 mL of 1,2-dimethoxyethane and 1 mL of saturated aqueous sodium bicarbonate. The mixture was heated at reflux for 3 h, then partitioned between dichloromethane and water. The combined organics were dried over sodium sulfate, concentrated and purified by column chromatography (eluting with EtOAc/Hexane, 2:1) to give 138 mg (64%) of 5-[4-(2-chloroethoxy)phenyl]-6-methyl-4-[(4-methyl-1H-indol-5-yl)amino]nicotinonitrile as an off-white solid, mp 200-201° C.; MS 417.2 (M+H).

### EXAMPLE 13

### Preparation of 5-[4-(2-CHLOROETHOXY)PHENYL]-4-(1H-INDOL-4-YLAMINO)-6-METHYLNICOTINONITRILE

Prepared from 5-iodo-6-methyl-4-(1H-indol-4-ylamino)nicotinonitrile and the boronic acid of 4-(2-chloroethoxy)bromo-benzene via the procedure used to prepare 5-[4-(2-chloroethoxy)phenyl]-6-methyl-4-[(4-methyl-1H-indol-5-yl)amino]nicotinonitrile, mp 185-186° C.; MS 403.3 (M+H).

### EXAMPLE 14

### Preparation of 6-METHYL-4-[(4-METHYL-1H-INDOL-5-YL) AMINO]-5-{4-[2-(4-METHYLPIPERAZIN-1-YL)ETHOXY]PHENYL}NICOTINONITRILE

A mixture of 5-[4-(2-chloroethoxy)phenyl]-6-methyl-4-[(4-methyl-1H-indol-5-yl)amino]nicotinonitrile (100 mg, 0.24 mmol) and N-methylpiperazine (240 mg, 2.3 mmol) in 5 mL of 1,2-dimethoxyethane containing a catalytic amount of sodium iodine was heated at reflux for 24 h, cooled to room temperature, diluted with aqueous sodium bicarbonate, filtered and washed with water to give a solid crude product. The crude product was purified by column chromatography, eluting with 5% methanol/dichloromethane, to give 74 mg (64%) of 6-methyl-4-[(4-methyl-1H-indol-5-yl)amino]-5-{4-[2-(4-methylpiperazin-1-yl)ethoxy]phenyl}nicotinonitrile as a white solid, mp 183-184° C.; MS 481.4 (M+H).

### EXAMPLE 15

### Preparation of 5-(5-FORMYL-1-BENZOFURAN-2-YL)-6-METHYL-4-[(4-METHYL-1H-INDOL-5-YL)AMINO]NICOTINONITRILE

### Step a): Preparation of 5-formylbenzofuran

To a −15° C. to −20° C. solution of 1-benzofuran-5-carbonitrile (5 g, 34.9 mmol) in 50 mL of dichloromethane under nitrogen was added DIBAL-H (41.9 mL, 41.9 mmol, 1.0 M in heptane) such that the temperature was less than or equal to −15° C. The reaction mixture was stirred for an additional 10 min at −15° C. to −20° C. and quenched by adding 2.0 N HCl dropwise such that the temperature was less than or equal to room temperature. The organic layer was then separated, washed with water, dried over sodium sulfate and concentrated to give 4 g (78%) of the title compound as a yellow oil, which was used in the next step without further purification.

### Step b): Preparation of 5-formyl-2-benzofurantributylstannane

To a solution of N-methylpiperazine (0.75 g, 7.5 mmol) in 15 mL of hexane at 0° C. under nitrogen was added dropwise a solution of n-BuLi (3.0 mL, 7.43 mmol, 2.5 M in hexanes). The reaction mixture was stirred at 0° C. for 40 min to give a thick white slurry. 5-Formylbenzofuran (1.0 g, 6.8 mmol) was added dropwise at 0° C. and the reaction mixture was stirred at 0° C. for 15 min. Tetramethylethylenendiamine (TMEDA) (1.7 g, 14.96 mmol) was then added in one portion followed by nBuLi (6.0 mL, 14.9 mmol, 2.5M in hexane) at 0° C. The reaction mixture was allowed to warm up to room temperature, stirred for 18 h and cooled to 0° C. After diluting the reaction mixture with 30 ml of tetrahydrofuran, the reaction mixture was cooled to −50° C. and tributyltin chloride (4.87 g, 15.0 mmol) was added dropwise. The resulting mixture was stirred at −50° C. for additional 15 min, stirred at room temperature for an extra 5-6 h, and partitioned between saturated aqueous sodium bicarbonate and diethyl ether. The combined organics were dried over sodium sulfate, concentrated and purified by column chromatography, eluting with 2% ethyl acetate/hexane, to give 1.0 g (34%) of the title compound as a yellow oil.

### Step c): Preparation of 5-(5-formyl-1-benzofuran-2-yl)-6-methyl-4-[(4-methyl-1H-indol-5-yl)amino]nicotinonitrile

A mixture of 5-iodo-6-methyl-4-[(4-methyl-1H-indol-5-yl)amino]nicotinonitrile (300 mg, 0.773 mmol), 5-formyl-2-benzofurantributylstannane (505 mg, 1.16 mmol) and (Ph₃P)₄Pd (45 mg, 0.0387 mmol) in 5 mL of DMF was heated at 120° C. for 3 h. The reaction mixture was partitioned between saturated aqueous sodium bicarbonate and dichloromethane. The combined organics were dried over sodium sulfate, concentrated and purified by column chromatography, eluting with ethyl acetate/hexane (1:1) to give 235 mg (75%) of 5-(5-formyl-1-benzofuran-2-yl)-6-methyl-4-[(4-methyl-1H-indol-5-yl)amino]nicotinonitrile as an off-white solid, mp 168-169° C.; MS 407.3 (M+H).

### EXAMPLE 16

### Preparation of 6-METHYL-4-[(4-METHYL-1H-INDOL-5-YL)AMINO]-5-{5-[(4-METHYLPIPERAZIN-1-YL)METHYL]-1-BENZOFURAN-2-YL}NICOTINONITRILE

Sodium cyanoborohydride (23 mg, 0.369 mmol) was added in portions to a stirred mixture of 5-(5-formyl-1-benzofuran-2-yl)-6-methyl-4-[(4-methyl-1H-indol-5-yl)amino]nicotinonitrile (100 mg, 0.246 mmol), N-methylpiperazine (50 mg, 0.493 mmol) and acetic acid (18 mg, 0.295 mmol) in 3 mL of ethanol. The reaction mixture was stirred at room temperature overnight, diluted with dichloromethane/methanol (10:1, 20 mL), adsorbed onto silica gel, and purified by column chromatography, eluting with 10% methanol/dichloromethane, to give 65 mg (54%) of 6-methyl-4-[(4-methyl-1H-indol-5-yl)amino]-5-{5-[(4-methylpiperazin-1-yl)methyl]-1-benzofuran-2-yl}nicotinonitrile as an off-white solid, mp 218-220° C.; MS 491.4 (M+H).

### EXAMPLES 17-42

The following compounds were prepared from 5-[4-(2-chloroethoxy)phenyl]-6-methyl-4-[(4-methyl-1H-indol-5-yl)amino]nicotinonitrile and the corresponding amine via the procedure set forth in Example 14, above.

| Example | | | HPLC |
|---|---|---|---|
| # | Compound Name | MS(M + H) | Results |
| 17 | 5-(4-{2-[(2-hydroxyethyl)amino]ethoxy}phenyl)-6- | 442.2 | 100% at |
| | methyl-4-[(4-methyl-1H-indol-5-yl)amino]nicotinonitrile | | 254 nm, |
| | | | 1.45 min. |
| 18 | 5-(4-{2-[(3-hydroxypropyl)amino]ethoxy}phenyl)-6- | 456.2 | 100% at |
| | methyl-4-[(4-methyl-1H-indol-5-yl)amino]nicotinonitrile | | 254 nm, |
| | | | 1.46 min. |
| 19 | 5-(4-{2-[(2-ethoxyethyl)amino]ethoxy}phenyl)-6- | 470.2 | 100% at |
| | methyl-4-[(4-methyl-1H-indol-5-yl)amino]nicotinonitrile | | 254 nm, |
| | | | 1.56 min. |
| 20 | 6-methyl-4-[(4-methyl-1H-indol-5-yl)amino]-5-[4-(2- | 452.2 | 100% at |
| | pyrrolidin-1-ylethoxy)phenyl]nicotinonitrile | | 254 nm, |
| | | | 1.52 min. |
| 21 | 6-methyl-4-[(4-methyl-1H-indol-5-yl)amino]-5-(4-{2- | 495.3 | 100% at |
| | [(2-pyrrolidin-1- | | 254 nm, |
| | ylethyl)amino]ethoxy}phenyl)nicotinonitrile | | 1.37 min. |
| 22 | 6-methyl-4-[(4-methyl-1H-indol-5-yl)amino]-5-(4-{2- | 495.3 | 100% at |
| | [(1-methylpiperidin-4- | | 254 nm, |
| | yl)amino]ethoxy}phenyl)nicotinonitrile | | 1.34 min. |
| 23 | 6-methyl-4-[(4-methyl-1H-indol-5-yl)amino]-5-[4-(2- | 509.3 | 95% at |
| | {[(1-methylpiperidin-4- | | 254 nm, |
| | yl)methyl]amino}ethoxy)phenyl]nicotinonitrile | | 1.36 min. |
| 24 | 6-methyl-4-[(4-methyl-1H-indol-5-yl)amino]-5-[4-(2- | 466.3 | 100% at |
| | piperidin-1-ylethoxy)phenyl]nicotinonitrile | | 254 nm, |
| | | | 1.56 min. |
| 25 | 5-(4-{2-[4-(2-hydroxyethyl)piperidin-1- | 510.3 | 100% at |
| | yl]ethoxy}phenyl)-6-methyl-4-[(4-methyl-1H-indol-5- | | 254 nm, |
| | yl)amino]nicotinonitrile | | 1.54 min. |
| 26 | 6-methyl-4-[(4-methyl-1H-indol-5-yl)amino]-5-{4-[2-(4- | 535.3 | 100% at |
| | pyrrolidin-1-ylpiperidin-1- | | 254 nm, |
| | yl)ethoxy]phenyl}nicotinonitrile | | 1.38 min. |
| 27 | 5-{4-[2-(1,4′-bipiperidin-1′-yl)ethoxy]phenyl}-6-methyl- | 549.3 | 100% at |
| | 4-[(4-methyl-1H-indol-5-yl)amino]nicotinonitrile | | 254 nm, |
| | | | 1.39 min. |
| 28 | 6-methyl-4-[(4-methyl-1H-indol-5-yl)amino]-5-{4-[2-(4- | 551.3 | 100% at |
| | morpholin-4-ylpiperidin-1- | | 254 nm, |
| | yl)ethoxy]phenyl}nicotinonitrile | | 1.37 min. |
| 29 | 6-methyl-4-[(4-methyl-1H-indol-5-yl)amino]-5-{4-[2-(4- | 542.3 | 96% at |
| | phenylpiperidin-1-yl)ethoxy]phenyl}nicotinonitrile | | 254 nm, |
| | | | 1.79 min. |
| 30 | 6-methyl-4-[(4-methyl-1H-indol-5-yl)amino]-5-[4-(2- | 468.2 | 100% at |
| | morpholin-4-ylethoxy)phenyl]nicotinonitrile | | 254 nm, |
| | | | 1.48 min. |
| 31 | 5-{4-[2-(2,5-dimethylpiperazin-1-yl)ethoxy]phenyl}-6- | 495.3 | 95% at |
| | methyl-4-[(4-methyl-1H-indol-5-yl)amino]nicotinonitrile | | 254 nm, |
| | | | 1.51 min. |
| 32 | 5-{4-[2-(3,5-dimethylpiperazin-1-yl)ethoxy]phenyl}-6- | 495.3 | 100% at |
| | methyl-4-[(4-methyl-1H-indol-5-yl)amino]nicotinonitrile | | 254 nm, |
| | | | 1.51 min. |
| 33 | 5-{4-[2-(4-ethylpiperazin-1-yl)ethoxy]phenyl}-6- | 495.3 | 92% at |
| | methyl-4-[(4-methyl-1H-indol-5-yl)amino]nicotinonitrile | | 254 nm, |
| | | | 1.52 min. |
| 34 | 5-{4-[2-(1,4-diazepan-1-yl)ethoxy]phenyl}-6-methyl-4- | 481.3 | 100% at |
| | [(4-methyl-1H-indol-5-yl)amino]nicotinonitrile | | 254 nm, |
| | | | 1.35 min. |
| 35 | 5-(4-{2-[4-(2-hydroxyethyl)piperazin-1- | 511.3 | 100% at |
| | yl]ethoxy}phenyl)-6-methyl-4-[(4-methyl-1H-indol-5- | | 254 nm, |
| | yl)amino]nicotinonitrile | | 1.49 min. |
| 36 | 5-[4-(2-{4-[2-(dimethylamino)ethyl]piperazin-1- | 538.4 | 100% at |
| | yl}ethoxy)phenyl]-6-methyl-4-[(4-methyl-1H-indol-5- | | 254 nm, |
| | yl)amino]nicotinonitrile | | 1.38 min. |
| 37 | 5-{4-[2-(4-cyclopentylpiperazin-1-yl)ethoxy]phenyl}-6- | 535.3 | 93% at |
| | methyl-4-[(4-methyl-1H-indol-5-yl)amino]nicotinonitrile | | 254 nm, |
| | | | 1.59 min. |
| 38 | 5-[4-(2-{[3-(1H-imidazol-1- | 506.3 | 89% at |
| | yl)propyl]amino}ethoxy)phenyl]-6-methyl-4-[(4-methyl- | | 254 nm, |
| | 1H-indol-5-yl)amino]nicotinonitrile | | 1.37 min. |
| 39 | 5-{4-[2-(benzylamino)ethoxy]phenyl}-6-methyl-4-[(4- | 488.2 | 100% at |
| | methyl-1H-indol-5-yl)amino]nicotinonitrile | | 254 nm, |
| | | | 1.65 min. |
| 40 | 6-methyl-4-[(4-methyl-1H-indol-5-yl)amino]-5-(4-{2- | 489.2 | 81% at |
| | [(pyridin-2- | | 254 nm, |
| | ylmethyl)amino]ethoxy}phenyl)nicotinonitrile | | 1.58 min. |
| 41 | 6-methyl-4-[(4-methyl-1H-indol-5-yl)amino]-5-(4-{2- | 489.2 | 70% at |
| | [(pyridin-3- | | 254 nm, |
| | ylmethyl)amino]ethoxy}phenyl)nicotinonitrile | | 1.49 min. |
| 42 | 6-methyl-4-[(4-methyl-1H-indol-5-yl)amino]-5-(4-{2- | 489.2 | 66% at |
| | [(pyridin-4- | | 254 nm, |
| | ylmethyl)amino]ethoxy}phenyl)nicotinonitrile | | 1.47 min. |

HPLC column: Aquasil C18, 5.0×0.21 cm column; conditions: 0.8 mL/min, 5.5 min gradient of acetonitrile in water/trifluoroacetic acid.

### EXAMPLE 43

### Preparation of 5-BROMO-4-(1H-INDOL-5-YLAMINO)-6-METHYLNICOTINONITRILE

A mixture of 5-bromo-4-chloro-6-methylnicotinonitrile (1.0 g, 4.31 mmol) and 5-aminoindole (830 mg, 6.29 mmol) in 10 mL of ethanol was heated at reflux for 2 h. The resulting mixture was cooled slightly and filtered washing with ethanol and diethyl ether. The solid was stirred with saturated aqueous sodium bicarbonate for 30 min then the aqueous mixture was extracted with ethyl acetate. The layers were separated and the organic layer was dried over magnesium sulfate, filtered and concentrated in vacuo. The residue was triturated with diethyl ether to give 852 mg (60%) of 5-bromo-4-(1H-indol-5-ylamino)-6-methylnicotinonitrile as a light tan solid, MS 337.2 (M+H); HPLC 99.9% at 215 nm, 8.6 min; Prodigy ODS3, 0.46×15 cm column, 1.0 mL/min, 20 min gradient of acetonitrile in water/trifluoroacetic acid.

### EXAMPLE 44

### Preparation of 5-BROMO-6-METHYL-4-[(4-METHYL-1H-INDOL-5-YL)AMINO]NICOTINONITRILE

A mixture of 5-bromo-4-chloro-6-methylnicotinonitrile (2.00 g, 8.63 mmol) and 5-amino-4-methylindole (1.82 mg, 12.47 mmol) in 20 mL of ethanol was heated at reflux overnight. The resulting mixture was cooled slightly and filtered washing with ethanol. The solid was stirred with saturated aqueous sodium bicarbonate then the aqueous mixture was extracted with ethyl acetate. The layers were separated and the organic layer was washed with saturated aqueous sodium bicarbonate, dried over magnesium sulfate, filtered and concentrated in vacuo. The residue was triturated with diethyl ether to give 1.21 g (41%) of 5-bromo-6-methyl-4-[(4-methyl-1H-indol-5-yl)amino]nicotinonitrile as an off-white solid, MS 341.2 (M+H); HPLC 99.2% at 215 nm, 9.5 min; Prodigy ODS3, 0.46×15 cm column, 1.0 mL/min, 20 min gradient of acetonitrile in water/trifluoroacetic acid.

### EXAMPLE 45

### Preparation of 5-BROMO-4-(1H-INDOL-6-YLAMINO)-6-METHYLNICOTINONITRILE

A mixture of 5-bromo-4-chloro-6-methylnicotinonitrile (500 mg, 2.15 mmol) and 6-aminoindole (415 mg, 3.14 mmol) in 5 mL of ethanol was heated at reflux for 2 h. The resulting mixture was cooled slightly and filtered washing with ethanol. The residue was poured into saturated aqueous sodium bicarbonate and the aqueous mixture was extracted with ethyl acetate. The layers were separated and the organic layer was dried over magnesium sulfate, filtered and concentrated in vacuo. The residue was triturated with diethyl ether to give 253 g (36%) of 5-bromo-4-(1H-indol-6-ylamino)-6-methylnicotinonitrile as an off-white solid, MS 327.1 (M+H); HPLC 99.7% at 215 nm, 9.1 min; Prodigy ODS3, 0.46×15 cm column, 1.0 mL/min, 20 min gradient of acetonitrile in water/trifluoroacetic acid.

### EXAMPLE 46

### Preparation of 5-(3,4-DIMETHOXYPHENYL)-4-(1H-INDOL-6-YLAMINO)-6-METHYLNICOTINONITRILE

A mixture of 5-bromo-4-(1H-indol-6-ylamino)-6-methylnicotinonitrile (109 mg, 0.33 mmol), 3,4-dimethoxyphenylboronic acid (100 mg, 0.55 mmol) and (Ph₃P)₄Pd (35 mg) in 10 mL of 1,2-dimethoxyethane and 5 mL of saturated aqueous sodium bicarbonate was heated at 90-100° C. for 4 h. After cooling to room temperature the reaction mixture was partitioned between saturated aqueous sodium bicarbonate and ethyl acetate. The organic layer was dried over magnesium sulfate, filtered and concentrated in vacuo. The residue was purified by flash column chromatography eluting with a gradient of 4:1 hexane:ethyl acetate to 1:1 hexane:ethyl acetate to give mg (29%) of 5-(3,4-dimethoxyphenyl)-4-(1H-indol-6-ylamino)-6-methyl nicotinonitrile as a light yellow solid, MS 385.3 (M+H); HPLC: 97.2% at 215 nm, 8.1 min.; Prodigy ODS3, 0.46×15 cm column, 1.0 mL/min, 20 min gradient of acetonitrile in water/trifluoroacetic acid.

### EXAMPLE 47

### Preparation of 5-IODO-4-(2-METHYL-1H-INDOL-5-YLAMINO)-6-METHYLNICOTINONITRILE

A mixture of 4-chloro-5-iodo-6-methylnicotinonitrile (1.0 g, 3.6 mmol) and 5-amino-2-methylindole (735 mg, 5 mmol) in 8 mL of ethanol was heated at reflux for 3 h. The resulting mixture was cooled to room temperature, diluted with saturated aqueous sodium bicarbonate, filtered and washed with ethanol, water to give 781 mg (56%) of 5-iodo-4-(2-methyl-1H-indol-5-ylamino)-6-methylnicotinonitrile as a light yellow solid, MS 389.1 (M+H); HPLC: 98.6% at 215 nm, 9.5 min.; Prodigy ODS3, 0.46×15 cm column, 1.0 mL/min, 20 min gradient of acetonitrile in water/trifluoroacetic acid.

### EXAMPLE 48

### Preparation of 5-(3,4-DIMETHOXYPHENYL)-4-(2-METHYL-1H-INDOL-5-YLAMINO)-6-METHYLNICOTINONITRILE

Prepared in 85% yield from 5-iodo-4-(2-methyl-1H-indol-5-ylamino)-6-methylnicotinonitrile and 3,4-dimethoxyphenylboronic acid via the procedure used to prepare 5-(3,4-dimethoxyphenyl)-4-(1H-indol-6-ylamino)-6-methylnicotinonitrile, MS 399.4 (M+H), HPLC: 93.8% at 215 nm, 8.2 min.; Prodigy ODS3, 0.46×15 cm column, 1.0 mL/min, 20 min gradient of acetonitrile in water/trifluoroacetic acid.

### EXAMPLE 49

### Preparation of 5-IODO-4-(1H-INDOL-5-YLAMINO)-6-METHYLNICOTINONITRILE

A mixture of 4-chloro-5-iodo-6-methylnicotinonitrile (3.9 g, 14 mmol) and 5-aminoindole (2.59 g, 19.6 mmol) in 32 mL of ethanol was heated at reflux for 6 h. The resulting mixture was cooled to room temperature, diluted with saturated aqueous sodium bicarbonate, filtered and washed with ethanol, water to give 1.75 g (34%) of 5-iodo-4-(1H-indol-5-ylamino)-6-methylnicotinonitrile as a tan solid, MS 375.0 (M+H); HPLC: 91.2% at 215 nm, 17.1 min.; Prodigy ODS3, 0.46×15 cm column, 1.0 mL/min, 20 min gradient of acetonitrile in water/trifluoroacetic acid.

### EXAMPLE 50-57

Compounds were prepared from 5-iodo-4-(1H-indol-5-ylamino)-6-methylnicotinonitrile and the corresponding boronic acid or ester via the procedure used to prepare 5-(3,4-dimethoxyphenyl)-4-(1H-indol-4-ylamino)-6-methylnicotinonitrile.

| Example | | | HPLC |
|---|---|---|---|
| # | Compound Name | MS(M + H) | Results |
| 50 | 4-(1H-indol-5-ylamino)-6-methyl-5- | 325.2 | 98.0% at 215 |
| | phenylnicotinonitrile | | nM, 7.8 min |
| 51 | 5-(3,4-dimethoxyphenyl)-4-(1H-indol-5-ylamino)- | 385.2 | 93.9% at 215 |
| | 6-methylnicotinonitrile | | nM, 7.6 min |
| 52 | 4-(1H-indol-5-ylamino)-5-(2-methoxyphenyl)-6- | 355.2 | 99.6% at 215 |
| | methylnicotinonitrile | | nM, 7.9 min |
| 53 | 4-(1H-indol-5-ylamino)-5-(3-methoxyphenyl)-6- | 355.2 | 99.7% at 215 |
| | methylnicotinonitrile | | nM, 8.2 min |
| 54 | 4-(1H-indol-5-ylamino)-5-(4-methoxyphenyl)-6- | 355.2 | 98.9% at 215 |
| | methylnicotinonitrile | | nM, 8.2 min |
| 55 | 5-(1-benzofuran-2-yl)-4-(1H-indol-5-ylamino)-6- | 365.2 | 96.0% at 215 |
| | methylnicotinonitrile | | nM, 9.9 min |
| 56 | 5-(1-benzothiophen-2-yl)-4-(1H-indol-5-ylamino)- | 381.2 | 99.1% at 215 |
| | 6-methylnicotinonitrile | | nM, 10.1 min |
| 57 | 5-{3-[(dimethylamino)methyl]phenyl}-4-(1H-indol- | 382.3 | 94.0% at 215 |
| | 5-ylamino)-6-methylnicotinonitrile | | nM, 4.3 min |

HPLC column: Prodigy ODS3, 0.46 × 15 cm column; conditions: 1.0 mL/min, 20 min gradient of acetonitrile in water/trifluoroacetic acid.

### EXAMPLE 58

### Preparation of 4-CHLORO-6-ETHYL-5-IODONICOTINONITRILE

To a −78° C. solution of 4-chloro-5-iodo-6-methylnicotinonitrile (278 mg, 1.0 mmol) in 3 mL of anhydrous THF was slowly added lithium bis(trimethylsilyl)amide (1.2 mL, 1.2 mmol). The resulting slurry was stirred at −78° C. for 1 h under N₂. Iodomethane (170 mg, 1.2 mmol) was added and the reaction mixture was stirred at −78° C. and then gradually warmed to room temperature overnight. The reaction was quenched with H₂O and then extracted three times with ethyl acetate. The combined organic layers were washed with H₂O and brine then dried over Na₂SO₄. After filtration and concentration, the residue was purified by CombiFlash (dichloromethane-hexane gradient), providing 167 mg (57%) of 4-chloro-6-ethyl-5-iodonicotinonitrile as a pale-yellow solid, MS 293 (M+H).

### EXAMPLE 59

### Preparation of 5-BROMO-4-CHLORO-6-ETHYLNICOTINONITRILE

To a −78° C. solution of 5-bromo-4-chloro-6-methylnicotinonitrile (392 mg, 1.7 mmol) in anhydrous tetrahydrofuran (8.0 mL) was slowly added lithium bis(trimethylsilyl)amide (3.8 mL, 3.8 mmol). The resulting slurry was stirred at −78° C. for 1 h under N₂. Iodomethane (483 mg, 3.4 mmol) was added and the reaction mixture was stirred at −78° C. then gradually warmed to room temperature overnight. The reaction was quenched with 1.0 M citric acid solution and H₂O, then extracted with ethyl acetate three times. The combined organic layers were washed with H₂O and brine then dried over Na₂SO₄. After filtration and concentration, the residue was purified by silica gel column chromatography, eluting with dichloromethane, providing 74 mg (18%) of 5-bromo-4-chloro 6-ethylnicotinonitrile as a yellow solid containing a small amount of 5-bromo-4-chloro-6-isopropyl-nicotinonitrile (<10%). MS 245 (M+H).

### EXAMPLE 60

### Preparation of 6-ETHYL-5-IODO-4-(4-METHYL-1H-INDOL-5-YLAMINO)NICOTINONITRILE

A reaction mixture of 4-chloro-6-ethyl-5-iodonicotinonitrile (160 mg, 0.55 mmol) and 5-amino-4-methylindole (80 mg, 0.55 mmol) in anhydrous ethanol (2.0 mL) was heated at reflux for 44 h. After cooling to room temperature, the reaction mixture was added to 10 mL of H₂O, then extracted three times with a 1:3 mixture of tetrahydrofuran: ethyl acetate. The combined organic layers were washed with H₂O, dried over Na₂SO₄, filtered and concentrated to provide 230 mg of crude 6-ethyl-5-iodo-4-(4-methyl-1H-indol-5-ylamino)nicotinonitrile as a dark solid which was used directly in the next reaction without further purification. MS 403.1 (M+H).

### EXAMPLE 61

### Preparation of 5-(3,4-DIMETHOXYPHENYL)-6-ETHYL-4-[(4-METHYL-1H-INDOL-5-YL)AMINO]NICOTINONITRILE

Crude 6-ethyl-5-iodo-4-(4-methyl-1H-indol-5-ylamino)nicotinonitrile (80 mg, 0.2 mmol), 3,4-dimethoxyphenylboronic acid (73 mg, 0.4 mmol) and Pd(PPh₃)₄ (45 mg, 0.04 mmol) were dissolved in 1.0 mL of 1,2-dimethoxyethane followed by the addition of 0.5 mL of saturated aqueous sodium bicarbonate. The reaction mixture was heated at reflux for 4 h. After cooling to room temperature, the reaction mixture was purified by silica gel column chromatography eluting with 9:1 dichloromethane: tetrahydrofuran providing 51 mg (62%) of 5-(3,4-dimethoxyphenyl)-6-ethyl-4-[(4-methyl-1H-indol-5-yl)amino]nicotinonitrile as a tan solid, MS 413.3 (M+H); HPLC: 88.7% at 215 nm, 11.4 min.; Prodigy ODS3, 0.46×15 cm column, 1.0 mL/min, 20 min gradient of methanol in water/trifluoroacetic acid.

### EXAMPLE 62

### Preparation of 5-BROMO-6-ETHYL-4-(4-METHYL-1H-INDOL-5-YLAMINO)-NICOTINONITRILE

A reaction mixture of 5-bromo-4-chloro-6-ethylnicotinonitrile (71 mg, 0.29 mmol) and 5-amino-4-methylindole (42 mg, 0.29 mmol) in anhydrous ethanol (1.0 mL) was heated at reflux for 48 h. After cooling to room temperature, the reaction mixture was added to 10 mL of H₂O and filtered. The solid residue was washed with H₂O and diethyl ether then dried in vacuo to provide a quantitative amount of crude 5-bromo-6-ethyl-4-(4-methyl-1H-indol-5-ylamino)-nicotinonitrile as a dark solid that was used directly without further purification. MS 355 (M+H).

### EXAMPLE 63

### Preparation of 4-[(7-CHLORO-4-METHYL-1H-INDOL-5-YL)AMINO]-5-IODO-6-METHYLNICOTINONITRILE

A solution of 7-chloro-4-methyl-1H-indol-5-amine (537 mg, 3.0 mmol) and 4-chloro-5-iodo-6-methylnicotinonitrile (1.0 g, 6.0 mmol) in ethanol (10 mL) was heated to reflux for 72 h. The reaction mixture was cooled to room temperature and filtered. The filter cake was washed with ethanol and dried in vacuo to provide 610 mg (82%) of 4-[(7-chloro-4-methyl-1H-indol-5-yl)amino]-5-iodo-6-methylnicotinonitrile as a yellow solid. MS 423.3 (M+H).

### EXAMPLE 64

### Preparation of 4-[(7-CHLORO-4-METHYL-1H-INDOL-5-YL)AMINO]-5-(3,4-DIMETHOXYPHENYL)-6-METHYLNICOTINONITRILE

Prepared from Example 63 and 3,4-dimethoxyphenylboronic acid by the procedure used to prepare Example 8, MS 433.1; HPLC retention time 8.4 min; Prodigy ODS3, 0.46×15 cm column, 1.0 mL/min, 20 min gradient acetonitrile in water/trifluoroacetic acid.

### EXAMPLE 65

### Preparation of 4-(1H-INDOL-5-YLAMINO)-6-METHYL-5-(2-THIENYL)NICOTINONITRILE

Prepared from 5-iodo-4-(1H-indol-5-ylamino)-6-methylnicotinonitrile (Example 49) and 2-thienylboronic acid by the procedure used to prepare Example 56, MS 331.2; HPLC retention time 10.8 min; Prodigy ODS3, 0.46×15 cm column, 1.0 mL/min, 20 min gradient methanol in water/trifluoroacetic acid.

### EXAMPLE 66

### Preparation of 5-{3-[(DIMETHYLAMINO)METHYL]PHENYL}-6-METHYL-4-[(4-METHYL-1H-INDOL-5-YL)AMINO]PYRIDINE-3-CARBONITRILE

Prepared from 5-iodo-6-methyl-4-[(4-methyl-1H-indol-5-yl)amino]nicotinonitrile (Example 7) and 3-(N,N-dimethylaminomethy)phenylboronic acid pinacol ester by the procedure used to prepare Example 57, MS 396.2; HPLC retention time 5.0 min; Prodigy ODS3, 0.46×15 cm column, 1.0 mL/min, 20 min gradient acetonitrile in water/trifluoroacetic acid.

### EXAMPLE 67

### Preparation of 5-(5-FORMYLFURAN-2-YL)-6-METHYL-4-[(4-METHYL-1H-INDOL-5-YL)AMINO]PYRIDINE-3-CARBONITRILE

A mixture of 5-iodo-6-methyl-4-[(4-methyl-1H-indol-5-yl)amino]nicotinonitrile (Example 7) (389 mg, 1.00 mmol), 5-formyl-2-furanboronic acid (235 mg, 1.68 mmol) and (Ph₃P)₄Pd (53 mg) in 8 mL of 1,2-dimethoxyethane and 4 mL of saturated aqueous sodium bicarbonate was heated at reflux for 30 min. After cooling to room temperature the reaction mixture was partitioned between aqueous sodium bicarbonate and ethyl acetate. The organic layer was dried over magnesium sulfate, filtered and concentrated in vacuo. The residue was purified by flash column chromatography eluting with a gradient of 4:1 hexane:ethyl acetate to 1:1 hexane:ethyl acetate. The fractions containing the desired product were combined, concentrated in vacuo and the residue triturated with diethyl ether and hexane to provide 49 mg of 5-(5-formylfuran-2-yl)-6-methyl-4-[(4-methyl-1H-indol-5-yl)amino]pyridine-3-carbonitrile as an orange solid, MS 357.1, HPLC retention time 7.1 min; Prodigy ODS3, 0.46×15 cm column, 1.0 mL/min, 20 min gradient acetonitrile in water/trifluoroacetic acid.

Concentration of the filtrate provided an additional 227 mg of 5-(5-formylfuran-2-yl)-6-methyl-4-[(4-methyl-1H-indol-5-yl)amino]pyridine-3-carbonitrile.

### EXAMPLE 68

### Preparation of 6-METHYL-4-[(4-METHYL-1H-INDOL-5-YL)AMINO]-5-{5-[(4-METHYLPIPERAZIN-1-YL)METHYL]FURAN-2-YL}PYRIDINE-3-CARBONITRILE

To a 0° C. mixture of 5-(5-formylfuran-2-yl)-6-methyl-4-[(4-methyl-1H-indol-5-yl)amino]pyridine-3-carbonitrile (Example 67) (225 mg, 0.632 mmol) and 1-methylpiperazine in 5 mL of dichloromethane and 1 mL of 1-methyl-2-pyrrolidinone was added sodium triacetoxyborohydride (670 mg). After stirring at 0° C. for 10 min, 2 drops of acetic acid were added and the reaction mixture was stirred at room temperature overnight. The reaction mixture was partitioned between aqueous sodium bicarbonate and ethyl acetate. The organic layer was dried over magnesium sulfate, filtered and concentrated in vacuo. The residue was purified by flash column chromatography eluting with a gradient of ethyl acetate to 10% methanol in ethyl acetate to 2% ammonium hydroxide in 10% methanol in ethyl acetate. The fractions containing the desired product were combined, concentrated in vacuo and the residue triturated with diethyl ether and hexane to provide 97 mg of 6-methyl-4-[(4-methyl-1H-indol-5-yl)amino]-5-{5-[(4-methyl piperazin-1-yl)methyl]furan-2-yl}pyridine-3-carbonitrile as an off-white solid, MS 441.2, HPLC retention time 4.6 min; Prodigy ODS3, 0.46×15 cm column, 1.0 mL/min, 20 min gradient acetonitrile in water/trifluoroacetic acid.

### EXAMPLE 69

### Preparation of 5-(5-FORMYLTHIOPHEN-2-YL)-6-METHYL-4-[(4-METHYL-1H-INDOL-5-YL)AMINO]PYRIDINE-3-CARBONITRILE

Prepared from 5-iodo-6-methyl-4-[(4-methyl-1H-indol-5-yl)amino]nicotinonitrile (Example 7) and 5-formyl-2-thiopheneboronic acid by the procedure used to prepare Example 67, MS 373.1; HPLC retention time 7.6 min; Prodigy ODS3, 0.46×15 cm column, 1.0 mL/min, 20 min gradient acetonitrile in water/trifluoroacetic acid.

### EXAMPLE 70

### Preparation of 6-METHYL-4-[(4-METHYL-1H-INDOL-5-YL)AMINO]-5-{5-[(4-METHYLPIPERAZIN-1-YL)METHYL]THIOPHEN-2-YL}PYRIDINE-3-CARBONITRILE

Prepared from 5-(5-formylthiophen-2-yl)-6-methyl-4-[(4-methyl-1H-indol-5-yl)amino]pyridine-3-carbonitrile (Example 69) and 1-methylpiperazine by the procedure used to prepare Example 67, MS 457.2.1; HPLC retention time 4.8 min; Prodigy ODS3, 0.46×15 cm column, 1.0 mL/min, 20 min gradient acetonitrile in water/trifluoroacetic acid.

### PKCθ IMAP Assay

The materials used include the following: human PKCθ full length enzyme (Panvera Cat# P2996); substrate peptide: 5FAM-RFARKGSLRQKNV-OH (Molecular Devices, RP7032); ATP (Sigma Cat # A2383); DTT (Pierce, 20291); 5× kinase reaction buffer (Molecular Devices, R7209); 5× binding buffer A (Molecular Devices, R7282), 5× binding buffer B (Molecular Devices, R7209); IMAP Beads (Molecular Devices, R7284); and 384-well plates (Corning Costar, 3710).

The reaction buffer was prepared by diluting the 5× stock reaction buffer and adding DTT to obtain a concentration of 3.0 mM. The binding buffer was prepared by diluting the 5× binding buffer A. A master mix solution was prepared using a 90% dilution of the reaction buffer containing 2×ATP (12 uM) and 2× peptide (200 nm). Compounds were diluted in DMSO to 20× of the maximum concentration for the IC₅₀ measurement. 27 μl of the master mix solution for each IC₅₀ curve was added to the first column in a 384-well plate and 3 μl of 20× compound in DMSO was added to each well.

The final concentration of compound was 2× and 10% DMSO. DMSO was added to the rest of the master mix to increase the concentration to 10%. 10 μl of the master mix containing 10% DMSO was added to the rest of the wells on the plate except the 2nd column. 20 μl was transferred from the first column to the 2nd column. The compounds were serially diluted in 2:1 ratio starting from the 2nd column. A 2×(2 nM) PKCθ solution was made in the reaction buffer. 10 μl of the PKCθ solution was added to every well to achieve these final concentrations: PKCθ-1 nM; ATP-6 μM; peptide-100 nM; DMSO-5%. Samples were incubated for 25 minutes at room temperature. The binding reagent was prepared by diluting the beads in 1× binding buffer to 800:1. 50 μl of the binding reagent was added to every well and incubated for 20 minutes. FP was measured using Envision2100 (PerkinElmer Life Sciences). Wells with no ATP and wells with no enzyme were used as controls.

The IC₅₀ results obtained are shown in Table I, below.

TABLE I

| | | PKC- |
|---|---|---|
| | | Theta |
| Example | | Mean |
| Number | Compound Name | IC50 (nM) |
| 8 | 5-(3,4-dimethoxyphenyl)-4-(1H-indol-4-ylamino)-6- | 151 |
| | methylnicotinonitrile | |
| 9 | 5-(3,4-dimethoxyphenyl)-6-methyl-4-[(4-methyl-1H-indol-5- | 67 |
| | yl)amino]nicotinonitrile | |
| 10 | 5-(1-benzofuran-2-yl)-4-(1H-indol-4-ylamino)-6-methylnicotinonitrile | 113 |
| 11 | 5-(1-benzofuran-2-yl)-6-methyl-4-[(4-methyl-1H-indol-5- | 13 |
| | yl)amino]nicotinonitrile | |
| 12 | 5-[4-(2-chloroethoxy)phenyl]-6-methyl-4-[(4-methyl-1H-indol-5- | 30 |
| | yl)amino]nicotinonitrile | |
| 13 | 5-[4-(2-chloroethoxy)phenyl]-4-(1H-indol-4-ylamino)-6- | 150 |
| | methylnicotinonitrile | |
| 14 | 6-methyl-4-[(4-methyl-1H-indol-5-yl)amino]-5-{4-[2-(4- | 31 |
| | methylpiperazin-1-yl)ethoxy]phenyl}nicotinonitrile | |
| 15 | 5-(5-formyl-1-benzofuran-2-yl)-6-methyl-4-[(4-methyl-1H-indol-5- | 12 |
| | yl)amino]nicotinonitrile | |
| 16 | 6-methyl-4-[(4-methyl-1H-indol-5-yl)amino]-5-{5-[(4-methylpiperazin- | 2.7 |
| | 1-yl)methyl]-1-benzofuran-2-yl}nicotinonitrile | |
| 17 | 5-(4-{2-[(2-hydroxyethyl)amino]ethoxy}phenyl)-6-methyl-4-[(4-methyl- | 19 |
| | 1H-indol-5-yl)amino]nicotinonitrile | |
| 18 | 5-(4-{2-[(3-hydroxypropyl)amino]ethoxy}phenyl)-6-methyl-4-[(4- | 40 |
| | methyl-1H-indol-5-yl)amino]nicotinonitrile | |
| 19 | 5-(4-{2-[(2-ethoxyethyl)amino]ethoxy}phenyl)-6-methyl-4-[(4-methyl- | 46 |
| | 1H-indol-5-yl)amino]nicotinonitrile | |
| 20 | 6-methyl-4-[(4-methyl-1H-indol-5-yl)amino]-5-[4-(2-pyrrolidin-1- | 25 |
| | ylethoxy)phenyl]nicotinonitrile | |
| 21 | 6-methyl-4-[(4-methyl-1H-indol-5-yl)amino]-5-(4-{2-[(2-pyrrolidin-1- | 26 |
| | ylethyl)amino]ethoxy}phenyl)nicotinonitrile | |
| 22 | 6-methyl-4-[(4-methyl-1H-indol-5-yl)amino]-5-(4-{2-[(1- | 9.0 |
| | methylpiperidin-4-yl)amino]ethoxy}phenyl)nicotinonitrile | |
| 23 | 6-methyl-4-[(4-methyl-1H-indol-5-yl)amino]-5-[4-(2-{[(1- | 8.0 |
| | methylpiperidin-4-yl)methyl]amino}ethoxy)phenyl]nicotinonitrile | |
| 24 | 6-methyl-4-[(4-methyl-1H-indol-5-yl)amino]-5-[4-(2-piperidin-1- | 44 |
| | ylethoxy)phenyl]nicotinonitrile | |
| 25 | 5-(4-{2-[4-(2-hydroxyethyl)piperidin-1-yl]ethoxy}phenyl)-6-methyl-4- | 68 |
| | [(4-methyl-1H-indol-5-yl)amino]nicotinonitrile | |
| 26 | 6-methyl-4-[(4-methyl-1H-indol-5-yl)amino]-5-{4-[2-(4-pyrrolidin-1- | 31 |
| | ylpiperidin-1-yl)ethoxy]phenyl}nicotinonitrile | |
| 27 | 5-{4-[2-(1,4′-bipiperidin-1′-yl)ethoxy]phenyl}-6-methyl-4-[(4-methyl- | 30 |
| | 1H-indol-5-yl)amino]nicotinonitrile | |
| 28 | 6-methyl-4-[(4-methyl-1H-indol-5-yl)amino]-5-{4-[2-(4-morpholin-4- | 28 |
| | ylpiperidin-1-yl)ethoxy]phenyl}nicotinonitrile | |
| 29 | 6-methyl-4-[(4-methyl-1H-indol-5-yl)amino]-5-{4-[2-(4-phenylpiperidin- | 272 |
| | 1-yl)ethoxy]phenyl}nicotinonitrile | |
| 30 | 6-methyl-4-[(4-methyl-1H-indol-5-yl)amino]-5-[4-(2-morpholin-4- | 124 |
| | ylethoxy)phenyl]nicotinonitrile | |
| 31 | 5-{4-[2-(2,5-dimethylpiperazin-1-yl)ethoxy]phenyl}-6-methyl-4-[(4- | 42 |
| | methyl-1H-indol-5-yl)amino]nicotinonitrile | |
| 32 | 5-{4-[2-(3,5-dimethylpiperazin-1-yl)ethoxy]phenyl}-6-methyl-4-[(4- | 25 |
| | methyl-1H-indol-5-yl)amino]nicotinonitrile | |
| 33 | 5-{4-[2-(4-ethylpiperazin-1-yl)ethoxy]phenyl}-6-methyl-4-[(4-methyl- | 44 |
| | 1H-indol-5-yl)amino]nicotinonitrile | |
| 34 | 5-{4-[2-(1,4-diazepan-1-yl)ethoxy]phenyl}-6-methyl-4-[(4-methyl-1H- | 14 |
| | indol-5-yl)amino]nicotinonitrile | |
| 35 | 5-(4-{2-[4-(2-hydroxyethyl)piperazin-1-yl]ethoxy}phenyl)-6-methyl-4- | 44 |
| | [(4-methyl-1H-indol-5-yl)amino]nicotinonitrile | |
| 36 | 5-[4-(2-{4-[2-(dimethylamino)ethyl]piperazin-1-yl}ethoxy)phenyl]-6- | 33 |
| | methyl-4-[(4-methyl-1H-indol-5-yl)amino]nicotinonitrile | |
| 37 | 5-{4-[2-(4-cyclopentylpiperazin-1-yl)ethoxy]phenyl}-6-methyl-4-[(4- | 30 |
| | methyl-1H-indol-5-yl)amino]nicotinonitrile | |
| 38 | 5-[4-(2-{[3-(1H-imidazol-1-yl)propyl]amino}ethoxy)phenyl]-6-methyl-4- | 14 |
| | [(4-methyl-1H-indol-5-yl)amino]nicotinonitrile | |
| 39 | 5-{4-[2-(benzylamino)ethoxy]phenyl}-6-methyl-4-[(4-methyl-1H-indol- | 73 |
| | 5-yl)amino]nicotinonitrile | |
| 40 | 6-methyl-4-[(4-methyl-1H-indol-5-yl)amino]-5-(4-{2-[(pyridin-2- | 29 |
| | ylmethyl)amino]ethoxy}phenyl)nicotinonitrile | |
| 41 | 6-methyl-4-[(4-methyl-1H-indol-5-yl)amino]-5-(4-{2-[(pyridin-3- | 64 |
| | ylmethyl)amino]ethoxy}phenyl)nicotinonitrile | |
| 42 | 6-methyl-4-[(4-methyl-1H-indol-5-yl)amino]-5-(4-{2-[(pyridin-4- | 41 |
| | ylmethyl)amino]ethoxy}phenyl)nicotinonitrile | |
| 46 | 5-(3,4-dimethoxyphenyl)-4-(1H-indol-6-ylamino)-6-methyl | 11900 |
| | nicotinonitrile | |
| 48 | 5-(3,4-dimethoxyphenyl)-6-methyl-4-[(2-methyl-1H-indol-5- | 556 |
| | yl)amino]nicotinonitrile | |
| 50 | 4-(1H-indol-5-ylamino)-6-methyl-5-phenylnicotinonitrile | 50 |
| 51 | 5-(3,4-dimethoxyphenyl)-4-(1H-indol-5-ylamino)-6-methyl | 31 |
| | nicotinonitrile | |
| 52 | 4-(1H-indol-5-ylamino)-5-(2-methoxyphenyl)-6-methylnicotinonitrile | 250 |
| 53 | 4-(1H-indol-5-ylamino)-5-(3-methoxyphenyl)-6-methylnicotinonitrile | 34 |
| 54 | 4-(1H-indol-5-ylamino)-5-(4-methoxyphenyl)-6-methylnicotinonitrile | 41 |
| 55 | 5-(1-benzofuran-2-yl)-4-(1H-indol-5-ylamino)-6-methylnicotinonitrile | 26 |
| 56 | 5-(1-benzothiophen-2-yl)-4-(1H-indol-5-ylamino)-6- | 137 |
| | methylnicotinonitrile | |
| 57 | 5-{3-[(dimethylamino)methyl]phenyl}-4-(1H-indol-5-ylamino)-6- | 87 |
| | methylnicotinonitrile | |
| 61 | 5-(3,4-dimethoxyphenyl)-6-ethyl-4-[(4-methyl-1H-indol-5- | 191 |
| | yl)amino]nicotinonitrile | |
| 64 | 4-[(7-chloro-4-methyl-1H-indol-5-yl)amino]-5-(3,4-dimethoxyphenyl)- | 84 |
| | 6-methylnicotinonitrile | |
| 65 | 4-(1H-indol-5-ylamino)-6-methyl-5-(2-thienyl)nicotinonitrile | 80 |
| 66 | 5-{3-[(dimethylamino)methyl]phenyl}-6-methyl-4-[(4-methyl-1H-indol- | 42 |
| | 5-yl)amino]pyridine-3-carbonitrile | |
| 67 | 5-(5-formylfuran-2-yl)-6-methyl-4-[(4-methyl-1H-indol-5-yl)amino] | 21 |
| | pyridine-3-carbonitrile | |
| 68 | 6-methyl-4-[(4-methyl-1H-indol-5-yl)amino]-5-{5-[(4-methylpiperazin- | 20 |
| | 1-yl)methyl]furan-2-yl}pyridine-3-carbonitrile | |
| 69 | 5-(5-formylthiophen-2-yl)-6-methyl-4-[(4-methyl-1H-indol-5- | 4.8 |
| | yl)amino]pyridine-3-carbonitrile | |
| 70 | 6-methyl-4-[(4-methyl-1H-indol-5-yl)amino]-5-{5-[(4-methylpiperazin- | 14 |
| | 1-yl)methyl]thiophen-2-yl}pyridine-3-carbonitrile | |

### Metabolic Stability

In order to probe the metabolic stability of the compounds of the invention, a series of compounds were selected for stability testing in rat liver microsomes. A DMSO solution of each compound was incubated for 30 minutes in the presence of rat liver homogenate. The percentage of remaining compound was assessed by HPLC. An approximate half life was calculated from the percentage of remaining compound. Table II, below, illustrates a comparison of the in vitro microsomal half life of the selected compounds verses a series of related compounds lacking the C-6 alkyl group. From these results, it is apparent that the C-6 alkyl group imparts a beneficial effect on the in vitro metabolic stability of the compounds of the invention. It is generally accepted in the industry that improved in vitro metabolic stability frequently translates into improved in vivo metabolic stability. Improved metabolic stability may translate to increased in vivo exposure levels.

TABLE II

| Rat Microsome Stability of 6-H cyanopyridine Compounds | | Rat Microsome Stability of 6-Me cyanopyridine Compounds | |
|---|---|---|---|
| 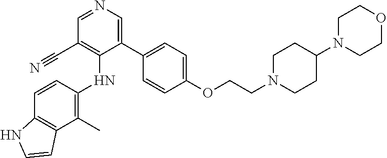 | 10 min | 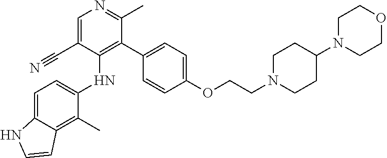 example 28 | |
| 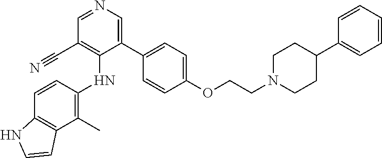 | 5 min | 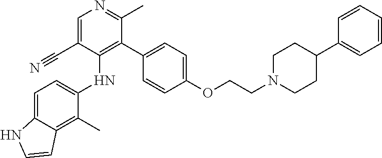 example 29 | 20 min |
| 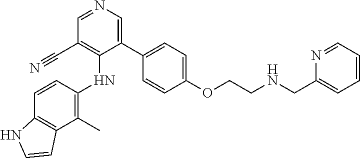 | 3 min | 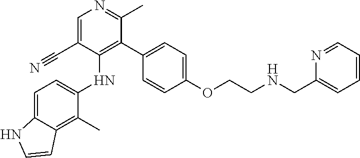 example 40 | 13 min |
| 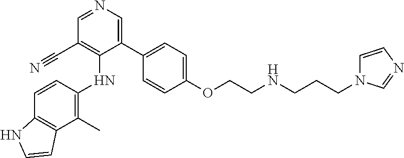 | 22 min | 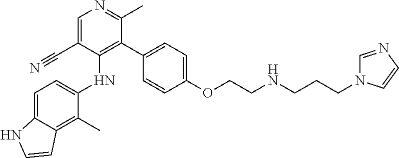 example 38 | >30 min |
| 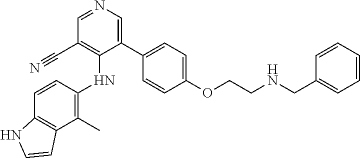 | 6 min | 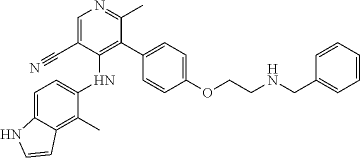 example 39 | 14 min |
| 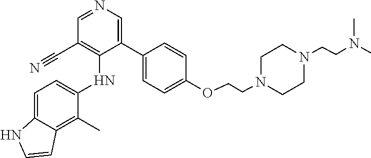 | >30 min | 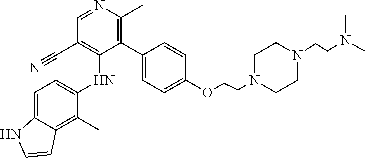 example 36 | >30 min |
| 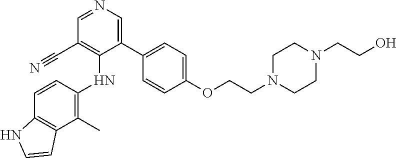 | 11 min | 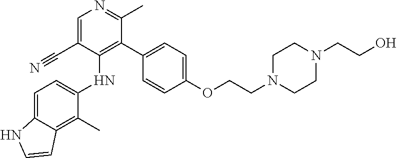 example 35 | >30 min |
| 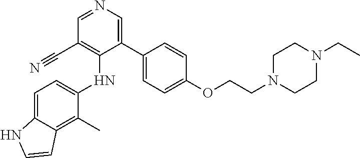 | 8 min | 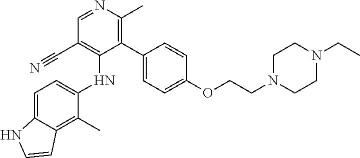 example 33 | >30 min |
| 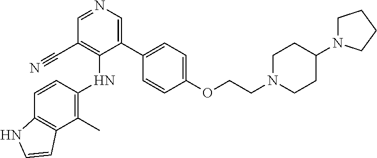 | >30 min | 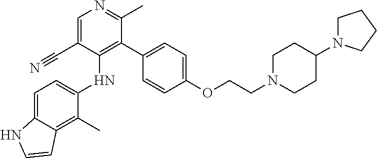 example 37 | >30 min |
| 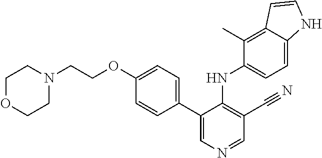 | 8 min | 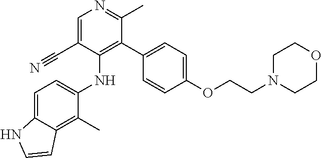 example 30 | 17 min |
| 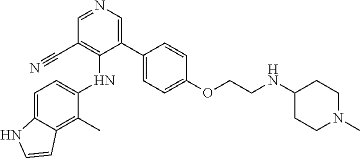 | >30 min | 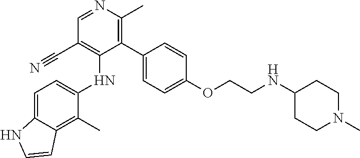 example 22 | >30 min |
| 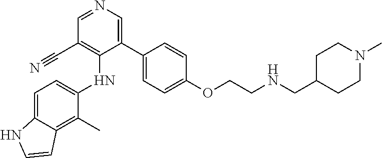 | >30 min | 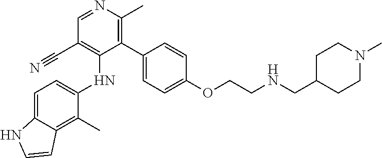 example 23 | >30 min |
| 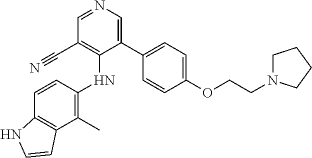 | 21 min | 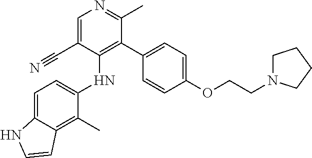 example 20 | >30 min |
| 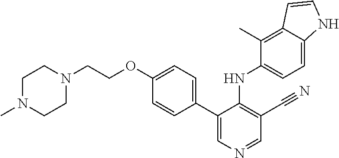 | 14 min | 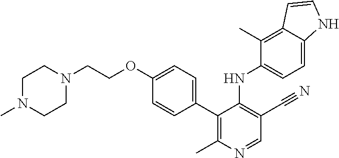 example 14 | >30 min |
| 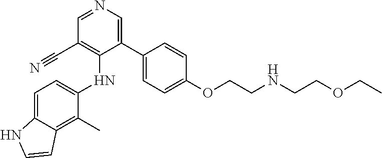 | 7 min | 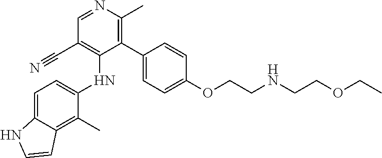 example 19 | >30 min |

Many variations of the present invention not illustrated herein will occur to those skilled in the art. The present invention is not limited to the embodiments illustrated and described herein, but encompasses all the subject matter within the scope of the appended claims.

What is claimed is:

1. A compound of formula I: 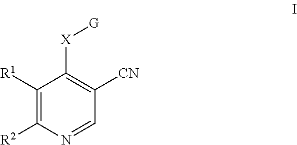
wherein G is 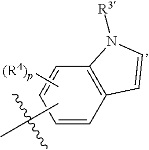
or a pharmaceutically acceptable salt thereof, wherein:
X is —O—, —N(R³)—, —S—, —S(O)— or —S(O)₂—;
R¹ is -A¹-(L)_{q1}-(A²-A³)_{q2}, wherein:
q1 and q2 are each independently 0 or 1;
A¹ is a C₆₋₁₀ aryl group or a 5- to 10-membered hetero₁₋₂aryl group, each of which is optionally substituted with 1 or 2 substituents independently selected from (a) halo, (b) —O—R³, (c) C₁₋₄ alkyl, (d) C₁₋₄ haloalkyl, (f) formyl, (g) —S—R³, (h) —N(R³)R³, (i) -Q-O—R³, (j) -Q-N(R³)—R³, (aa) —O-Q-O—R³, (bb) —O-Q-N(R³)R³, (cc) —N(R³)R³-Q-N(R³)R³, (dd) —N(R³)R³-Q-OR³, (p) —C(O)—O—R³, (q) —O—C(O)—R³, (r) —C(O)—N(R³)—R³, (s) —N(R³)—C(O)—R³, (t) —N(R³)—S(O)₂—R³, and (u) —S(O)₂N(R³)—R³; wherein Q, at each occurrence, is independently C₁₋₄ alkylene,
L is —(Y¹)ₙ₁—Z¹-(Y²)ₙ₂—(Z²)ₙ₃—, wherein:
n1, n2 and n3 are each independently 0 or 1,
provided that when n2 is 0, then n3 is also 0;
Y¹ and Y² are each independently —O—, —N(R³)—, —S—, —S(O)—, —S(O)₂—, —C(O)—O—, —O—C(O)—, —C(O)—N(R³)—, —N(R³)—C(O)—, —N(R³)—S(O)₂—, or —S(O)₂—N(R³)—; and
Z¹ and Z² are each independently C₁₋₄ alkylene;
A² is (a) halo, (b) —O—R³, (g) —S—R³, (h) —N(R³)R³, (k) phenyl, (l) 5- or 6-membered hetero₁₋₂aryl (m) 3- to 8-membered hetero₁₋₂cyclyl, (ee) Q-(3- to 8-membered hetero₁₋₂cyclyl), (ff)-C(O)-(3- to 8-membered hetero₁₋₂cyclyl), (gg) C₂₋₄ alkene or (hh) C₂₋₄alkyne, wherein each of (k)-(m), (ee)-(hh) is optionally substituted with 1 or 2 substituents independently selected from (a) halo, (b) —O—R³, (c) C₁₋₄ alkyl, (d) C₁₋₄ haloalkyl, (g) —S—R³, (h) —N(R³)R³, (i) -Q-O—R³, (j) -Q-N(R³)—R³, (q) —O—C(O)—R³, (r) —C(O)—N(R³)—R³, (s) —N(R³)—C(O)—R³, (t) —N(R³)—S(O)₂—R³, and (u) —S(O)₂—N(R³)—R³; wherein Q, at each occurrence, is independently C₁₋₄ alkylene, (p) —C(O)—O—R³, and
A³ is (e) H, (k) phenyl, (m) 3- to 8-membered hetero₁₋₂cyclyl, (n) C₃₋₈ cycloalkyl, or (o) an electron pair, wherein each of (k), (m) and (n) is optionally substituted with 1 or 2 substituents independently selected from (a) halo, (b) —O—R³, (c) C₁₋₄ alkyl, (d) C₁₋₄ haloalkyl, (g) —S—R³, (h) —N(R³)R³, (i) -Q-O—R³. (j) -Q-N(R³)—R³, (p) —C(O)—O—R³, (q) —O—C(O)—R³, (r) —C(O)—N(R³)—R³, (s) —N(R³)—C(O)—R³, (t) —N(R³)—S(O)₂—R³, and (u) —S(O)₂—N(R³)—R³, wherein Q, at each occurrence, is independently C₁₋₄ alkylene,
provided that when A² is (a) halo, (b) —O—R³, (g) —S—R³ or (h) —N(R³)R³, then A³ is (o) an electron pair on the halogen atom or heteroatom;
R² is (c) C₁₋₄ alkyl or (d) —CF₃;
R³, at each occurrence, is independently selected from (e) H and (c) C₁₋₄ alkyl;
R^{3′} is (e) H or (c) C₁₋₄ alkyl;
R⁴, at each occurrence, is independently selected from (a) halogen, (b) —O—R³ (c) C₁₋₄ alkyl, (d) —CF₃, and (h) —N(R³)—R³; and
p is 0, 1 or 2.

2. The compound of claim 1, or a pharmaceutically acceptable salt thereof, wherein X is —N(R³)—.

3. The compound of claim 2, or a pharmaceutically acceptable salt thereof, wherein X is —NH—.

4. The compound of any of claims 1-3, or a pharmaceutically acceptable salt thereof, wherein R² is C₁₋₄ alkyl.

5. The compound of claim 4, or a pharmaceutically acceptable salt thereof, wherein R² is methyl or ethyl.

6. The compound of any of claims 1-5, or a pharmaceutically acceptable salt thereof, wherein R^{3′} is H.

7. The compound of any of claims 1-6, or a pharmaceutically acceptable salt thereof, wherein p is 0-2 and R⁴ is C₁₋₄ alkyl.

8. The compound of claim 7, or a pharmaceutically acceptable salt thereof, wherein R⁴ is methyl.

9. The compound of any of claims 1-5, or a pharmaceutically acceptable salt thereof, wherein G is

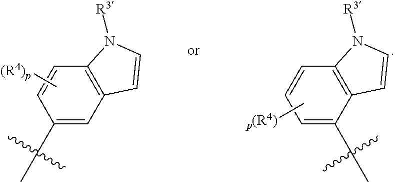

10. The compound of claim 9, or a pharmaceutically acceptable salt thereof, wherein G is indol-5-yl, 2-methylindol-5-yl, 4-methylindol-5-yl, 7-chloro-4-methyl-5-yl or indol-4-yl.

11. The compound of any of claims 1-10, or a pharmaceutically acceptable salt thereof, wherein A¹ is a C₆₋₁₀ aryl group optionally substituted with 1 or 2 substituents.

12. The compound of claim 1, or a pharmaceutically acceptable salt thereof, wherein A¹ is phenyl optionally substituted with 1 or 2 substituents.

13. The compound of any of claims 1-10, or a pharmaceutically acceptable salt thereof, wherein A¹ is a a 5- to 10-membered hetero₁₋₂aryl group, optionally substituted with 1 or 2 substituents.

14. The compound of claim 13, or a pharmaceutically acceptable salt thereof, wherein the 5- to 10-membered hetero₁₋₂aryl group is furanyl, thiophenyl, benzofuranyl, benzothienyl or indolyl, each optionally substituted with 1 or 2 substituents.

15. The compound of any of claims 1-14, or a pharmaceutically acceptable salt thereof, wherein:
n2 and n3 are each 0;
Y¹ is —O— or —N(R³)—; and
Z¹ is methylene, ethylene or trimethylene, or when q2 is 0, Z¹ is methyl, ethyl or propyl.

16. The compound of any of claims 1-14, or a pharmaceutically acceptable salt thereof, wherein:
n2 and n3 are each 1;
Y¹ is —O— or —N(R³)—;
Z¹ is methylene, ethylene or trimethylene;
Y² is —O— or —N(R³)—; and
Z² is methylene, ethylene or trimethylene, or when q2 is 0, Z² is methyl, ethyl or propyl.

17. The compound of any of claims 1-14, or a pharmaceutically acceptable salt thereof, wherein:
q1 and q2 are each 1; and
n1, n2 and n3 are each 0.

18. The compound of any of claims 1-14, or a pharmaceutically acceptable salt thereof, wherein:
A² is (a) halo, (b) —O—R³, (k) phenyl, (l) 5- or 6-membered hetero₁₋₂aryl, or (m) 5- to 7-membered hetero₁₋₂cyclyl; and
A³ is (e) H, (k) phenyl, (m) 5- to 7-membered hetero₁₋₂cyclyl, (n) C₅₋₇ cycloalkyl or (o) an electron pair;
wherein each of (k)-(n) is independently optionally substituted with 1 or 2 substituents.

19. The compound of claim 18, or a pharmaceutically acceptable salt thereof, wherein:
A² is (a) halo, (b) —O—R³, (k) phenyl, (l1) imidazolyl, (l2) pyridinyl, (m1) pyrrolidinyl, (m2) piperidinyl, (m3) piperazinyl, (m4) morpholinyl or (m5) 1,4-diazepanyl; and
A³ is (e) H, (k) phenyl, (m1) pyrrolidinyl, (m2) piperidinyl, (m3) morpholinyl, (n) cyclopentyl or (o) an electron pair;
wherein each of (k)-(n) is independently optionally substituted with 1 or 2 substituents independently selected from (b) —O—R³, (c) C₁₋₄ alkyl, (i) -Q-O—R³ and (j) -Q-N(R³)—R³.

20. A compound, or a pharmaceutically acceptable salt thereof, wherein the compound is selected from:
5-(3,4-dimethoxyphenyl)-4-(1H-indol-4-ylamino)-6-methylnicotinonitrile;
5-(3,4-dimethoxyphenyl)-6-methyl-4-[(4-methyl-1H-indol-5-yl)amino]nicotinonitrile;
5-(1-benzofuran-2-yl)-4-(1H-indol-4-ylamino)-6-methylnicotinonitrile;
5-(1-benzofuran-2-yl)-6-methyl-4-[(4-methyl-1H-indol-5-yl)amino]nicotinonitrile;
5-[4-(2-chloroethoxy)phenyl]-6-methyl-4-[(4-methyl-1H-indol-5-yl)amino]nicotinonitrile;
5-[4-(2-chloroethoxy)phenyl]-4-(1H-indol-4-ylamino)-6-methylnicotinonitrile;
6-methyl-4-[(4-methyl-1H-indol-5-yl)amino]-5-{4-[2-(4-methylpiperazin-1-yl)ethoxy]phenyl}nicotinonitrile;
5-(5-formyl-1-benzofuran-2-yl)-6-methyl-4-[(4-methyl-1H-indol-5-yl)amino]nicotinonitrile;
6-methyl-4-[(4-methyl-1H-indol-5-yl)amino]-5-{5-[(4-methylpiperazin-1-yl)methyl]-1-benzofuran-2-yl}nicotinonitrile;
5-(4-{2-[(2-hydroxyethyl)amino]ethoxy}phenyl)-6-methyl-4-[(4-methyl-1H-indol-5-yl)amino]nicotinonitrile;
5-(4-{2-[(3-hydroxypropyl)amino]ethoxy}phenyl)-6-methyl-4-[(4-methyl-1H-indol-5-yl)amino]nicotinonitrile;
5-(4-{2-[(2-ethoxyethyl)amino]ethoxy}phenyl)-6-methyl-4-[(4-methyl-1H-indol-5-yl)amino]nicotinonitrile;
6-methyl-4-[(4-methyl-1H-indol-5-yl)amino]-5-[4-(2-pyrrolidin-1-ylethoxy)phenyl]nicotinonitrile;
6-methyl-4-[(4-methyl-1H-indol-5-yl)amino]-5-(4-{2-[(2-pyrrolidin-1-ylethyl)amino]ethoxy}phenyl)nicotinonitrile;
6-methyl-4-[(4-methyl-1H-indol-5-yl)amino]-5-(4-{2-[(1-methylpiperidin-4-yl)amino]ethoxy}phenyl)nicotinonitrile;
6-methyl-4-[(4-methyl-1H-indol-5-yl)amino]-5-[4-(2-{[(1-methylpiperidin-4-yl)methyl]amino}ethoxy)phenyl]nicotinonitrile;
6-methyl-4-[(4-methyl-1H-indol-5-yl)amino]-5-[4-(2-piperidin-1-ylethoxy)phenyl]nicotinonitrile;
5-(4-{2-[4-(2-hydroxyethyl)piperidin-1-yl]ethoxy}phenyl)-6-methyl-4-[(4-methyl-1H-indol-5-yl)amino]nicotinonitrile;
6-methyl-4-[(4-methyl-1H-indol-5-yl)amino]-5-{4-[2-(4-pyrrolidin-1-ylpiperidin-1-yl)ethoxy]phenyl}nicotinonitrile;
5-{4-[2-(1,4′-bipiperidin-1′-yl)ethoxy]phenyl}-6-methyl-4-[(4-methyl-1H-indol-5-yl)amino]nicotinonitrile;
6-methyl-4-[(4-methyl-1H-indol-5-yl)amino]-5-{4-[2-(4-morpholin-4-ylpiperidin-1-yl)ethoxy]phenyl}nicotinonitrile;
6-methyl-4-[(4-methyl-1H-indol-5-yl)amino]-5-{4-[2-(4-phenylpiperidin-1-yl)ethoxy]phenyl}nicotinonitrile;
6-methyl-4-[(4-methyl-1H-indol-5-yl)amino]-5-[4-(2-morpholin-4-ylethoxy)phenyl]nicotinonitrile;
5-{4-[2-(2,5-dimethylpiperazin-1-yl)ethoxy]phenyl}-6-methyl-4-[(4-methyl-1H-indol-5-yl)amino]nicotinonitrile;
5-{4-[2-(3,5-dimethylpiperazin-1-yl)ethoxy]phenyl}-6-methyl-4-[(4-methyl-1H-indol-5-yl)amino]nicotinonitrile;
5-{4-[2-(4-ethylpiperazin-1-yl)ethoxy]phenyl}-6-methyl-4-[(4-methyl-1H-indol-5-yl)amino]nicotinonitrile;
5-{4-[2-(1,4-diazepan-1-yl)ethoxy]phenyl}-6-methyl-4-[(4-methyl-1H-indol-5-yl)amino]nicotinonitrile;
5-(4-{2-[4-(2-hydroxyethyl)piperazin-1-yl]ethoxy}phenyl)-6-methyl-4-[(4-methyl-1H-indol-5-yl)amino]nicotinonitrile;
5-[4-(2-{4-[2-(dimethylamino)ethyl]piperazin-1-yl]ethoxy)phenyl}-6-methyl-4-[(4-methyl-1H-indol-5-yl)amino]nicotinonitrile;
5-{4-[2-(4-cyclopentylpiperazin-1-yl)ethoxy]phenyl}-6-methyl-4-[(4-methyl-1H-indol-5-yl)amino]nicotinonitrile;
5-[4-(2-{[3-(1H-imidazol-1-yl)propyl]amino}ethoxy)phenyl]-6-methyl-4-[(4-methyl-1H-indol-5-yl)amino]nicotinonitrile;
5-{4-[2-(benzylamino)ethoxy]phenyl}-6-methyl-4-[(4-methyl-1H-indol-5-yl)amino]nicotinonitrile;
6-methyl-4-[(4-methyl-1H-indol-5-yl)amino]-5-(4-{2-[(pyridin-2-ylmethyl)amino]ethoxy}phenyl)nicotinonitrile;
6-methyl-4-[(4-methyl-1H-indol-5-yl)amino]-5-(4-{2-[(pyridin-3-ylmethyl)amino]ethoxy}phenyl)nicotinonitrile;
6-methyl-4-[(4-methyl-1H-indol-5-yl)amino]-5-(4-{2-[(pyridin-4-ylmethyl)amino]ethoxy}phenyl)nicotinonitrile;
5-(3,4-dimethoxyphenyl)-4-(1H-indol-6-ylamino)-6-methylnicotinonitrile;
5-(3,4-dimethoxyphenyl)-4-(2-methyl-1H-indol-5-ylamino)-6-methylnicotinonitrile;
4-(1H-indol-5-ylamino)-6-methyl-5-phenylnicotinonitrile;
5-(3,4-dimethoxyphenyl)-4-(1H-indol-5-ylamino)-6-methylnicotinonitrile;
4-(1H-indol-5-ylamino)-5-(2-methoxyphenyl)-6-methylnicotinonitrile;
4-(1H-indol-5-ylamino)-5-(3-methoxyphenyl)-6-methylnicotinonitrile;
4-(1H-indol-5-ylamino)-5-(4-methoxyphenyl)-6-methylnicotinonitrile;
5-(1-benzofuran-2-yl)-4-(1H-indol-5-ylamino)-6-methylnicotinonitrile;
5-(1-benzothiophen-2-yl)-4-(1H-indol-5-ylamino)-6-methylnicotinonitrile;
5-{3-[(dimethylamino)methyl]phenyl}-4-(1H-indol-5-ylamino)-6-methyl nicotinonitrile;
5-(3,4-dimethoxyphenyl)-6-ethyl-4-[(4-methyl-1H-indol-5-yl)amino]nicotinonitrile;
4-[(7-chloro-4-methyl-1H-indol-5-yl)amino]-5-(3,4-dimethoxyphenyl)-6-methyl nicotinonitrile;
4-(1H-indol-5-ylamino)-6-methyl-5-(2-thienyl)nicotinonitrile;
5-{3-[(dimethylamino)methyl]phenyl}-6-methyl-4-[(4-methyl-1H-indol-5-yl)amino]pyridine-3-carbonitrile;
5-(5-formylfuran-2-yl)-6-methyl-4-[(4-methyl-1H-indol-5-yl)amino]pyridine-3-carbonitrile;
6-methyl-4-[(4-methyl-1H-indol-5-yl)amino]-5-{5-[(4-methylpiperazin-1-yl)methyl]furan-2-yl}pyridine-3-carbonitrile;
5-(5-formylthiophen-2-yl)-6-methyl-4-[(4-methyl-1H-indol-5-yl)amino]pyridine-3-carbonitrile; and
6-methyl-4-[(4-methyl-1H-indol-5-yl)amino]-5-{5-[(4-methylpiperazin-1-yl)methyl]thiophen-2-yl}pyridine-3-carbonitrile.

21. A compound of any of claims 1-20, or a pharmaceutically acceptable salt thereof, for use in treating or inhibiting a pathological condition or disorder mediated by a protein kinase in a mammal.

22. A composition comprising a compound of any of claims 1-20, or a pharmaceutically acceptable salt thereof, and one or more pharmaceutically acceptable excipients.

23. A method of treating or inhibiting a pathological condition or disorder mediated by a protein kinase in a mammal, comprising administering to the mammal a therapeutically effective amount of the compound of any of claims 1-20, or a pharmaceutically acceptable salt thereof.

24. The method of claim 23, wherein the protein kinase is protein kinase C.

25. The method of claim 24 wherein the protein kinase C is a theta isoform.

26. The method of claim 23, wherein the pathological condition or disorder is selected from asthma, colitis, multiple sclerosis, psoriasis, arthritis, rheumatoid arthritis, inflammatory bowel disease, and joint inflammation.

27. A process for preparing a compound of formula I as defined in claim 1, said process comprising reacting a compound of formula xii with a compound of formula II, 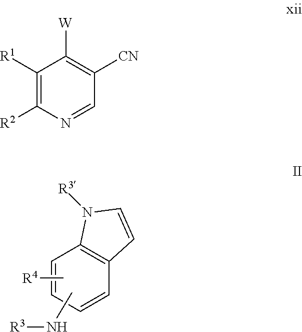
wherein R¹, R², R³ and R⁴ are as defined in claim 1, and W is CL or F.

28. The process of claim 27 further comprising reacting CsF with a compound of formula xii wherein W is CL, to form a compound of formula xii wherein W is F.

29. The process according to claim 27 or claim 28 further comprising reacting a compound of formula xi 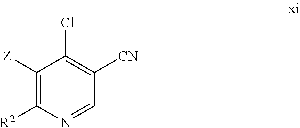
wherein Z is I or Br, and wherein R² is as defined in claim 1;
with a compound selected from the group consisting of R¹B(OH)₂, R¹B(OR)₂, and R¹SnR₃, wherein each R independently is a C₁-C₄alkyl group, to form the compound of formula xii in which W is Cl.

30. A process for preparing a compound of formula I as defined in claim 1, said process comprising reacting a compound of formula viii, wherein Z is I or Br, and R², R³ and R⁴ are as defined in claim 1, 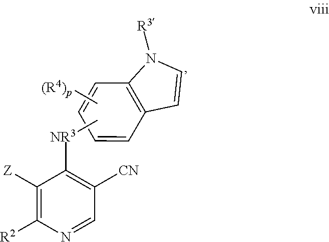
with a compound selected from the group consisting of R¹B(OH)₂, R¹B(OR)₂, and with R¹SnR₃, wherein each R independently is a C₁-C₄ alkyl group.

31. The process of claim 30 further comprising reacting a compound of formula II as defined in claim 27, with a compound of formula xi, as defined in claim 29, to form the compound of formula viii.
